SEQUENCE LISTING (355 sequences)

SEQ ID NO: 1 - DNA, 442 bp, Artificial Sequence
  Synthetic
gtggttccttgaaactctccctgtagaccgtcatgtgatccacatgcagttagggacactgaggccagggtagggaagtacccagcaaggaatgcagagagagccgctgagagcagcacctttgggttcccgattctgcttcaggacctggactttggtttaactttcctcaacttgcatttggcagattagaaaagatactcaaagcaacaagttatttcaggcctagctgacctttaataaaaagcacatatgggctgggtgcagtgactcatgcctgtaatcccagcactttgggaggctgagataggcagatcacttgaggtcaggagttcaagaccagcctggccaacatggtgaaaccttatctctactaaaaatacaaaaaattaaccaggcatggtggcacacatctgtagtcccagctactcgggagggtgag

SEQ ID NO: 2 - DNA, 122 bp, Artificial Sequence
  Synthetic
atgaagtcccagcaagaaatgctttatcagacacaagtgactgcatttgttcaagaacctagaagttggagaaacagcacctggatttgtatactctgagtatgaaaaagagtatgaaaaag

SEQ ID NO: 3 - DNA, 122 bp, Artificial Sequence
  Synthetic
atcccttactagggaccgagggggatgagaatgtgctttagcactttggccagaaatgagagaagacttctaacatgattgaaaccatggcctccaggaaaagactgaatttcaaatgtgac

SEQ ID NO: 4 - DNA, 122 bp, Artificial Sequence
  Synthetic
atgaagctgtcttcaaatgttagttctgcttcgtaaactagctcaatgctgaaactgtaaagtacccaaaagttactatgccccgaagttaaatatgtatagcctacttacatttacttaag

SEQ ID NO: 5 - DNA, 512 bp, Artificial Sequence
  Synthetic
ccttgcctgcaccatgaagttgagggagggagaaggcctggctctcccggagtcaggcaggcggcagtgggacccagagcccaggatgctgccaggccaagcagcagcaacactcaccgccgtgtggctggctttgccggtgaggatgctgcgggccttctttttggtcatgttgaagtttttcaggtaggcattgtagatgtgcttggagaaggccttcaggtcggccacctgtgggttgtactggctcccctcagtttgcagtcagccctgccaccagcttcctcttctcagcctccggcatccgaccaaaacggatagctgcacagggaagggggcagtcagcaaggagcccaggcaggccccagcacctctgacatccccatccctttacaggtgcatgggccaaatccttttggagcctaaggcccccagaagctctagagtcagcaaggtaggaatgatggggtggcccctccagatcgcaagctccacaagatgtgatttttttt

SEQ ID NO: 6 - DNA, 122 bp, Artificial Sequence
  Synthetic
atacatagatagatacatagataaaattgcaattccatcttctagatcctatgatgggctacaggagcccagtgtcctctcccatcccatagacagcccccctacccagaattgtaatgaac

SEQ ID NO: 7 - DNA, 122 bp, Artificial Sequence
  Synthetic
tccaacaaaatgaggggttatgctgttctgcatagtatttcaaacatttgtcttcattgcacggagctatattcctgtaaattgatttctttaggataatatttctttcttaaatatggctt

SEQ ID NO: 8 - DNA, 494 bp, Artificial Sequence
  Synthetic
ggatggataggatgacaggaaagctggccccaaattctgccacccacaactttaggctcctggggcatagggatgggaggaaaaccccagttcccgagtgctgggctggaagacaggaggccggggttcttgtgtcaggactgcccaggactggtgggtggcctggggcacactgctgccctttctgttgcctgtggtaagtgggggacaccagctgacacttcctgcctgtcgtccccagagcctgctgacagcgcacgatcagttcaaggcaacactgcccgaggctgacctgagagcgaggtgccatcatgggcatccagggtgagatccagaagatctgccagacgtatgggctgcggccctgctccaccaatccctacatcaccctcagcccgcaggacatcaacaccaagtgggatatggtcagtgccacctgcagccttcctcccaccccctcctgcatactgtgaccaccctgaaatctcgggtgg

SEQ ID NO: 9 - DNA, 122 bp, Artificial Sequence
  Synthetic
ctttttttttttttccaatgtccagcctaaactataaagaactttgagaacgcacagtgaagccataagcttgccaataaagagtcctctgtggtatggaactggcttatttcatacacaat

SEQ ID NO: 10 - DNA, 770 bp, Artificial Sequence
  Synthetic
aaaaaaaaaaaaaaaggagaggagagagactcaagcacgcccctcacaggactgctgaggccctgcaggtgtctgcagcatgtgccccaggccggggactctgtaagccactgctggagaccactcccatcctttctcccatttctctagacctgctgcctatacagtcactttttttttttttttgagacggagtctcgctctgtcgcccaggctggagtgcagtggcgggatctcggctcactgcaagctccgcctcccgggttcacgccattctcctgcctcagcctcccaagtagctgggaccacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgatacagtcacttttatgtggtttcgccaattttattccagctctgaaattctctgagctccccttacaagcagaggtgagctaagggctggagctcaaggcattcaaacccctaccagatctgacgaatgtgatggccacatcccggaaatatgaagacctgttatgggcatgggagggctggcgagacaaggcggggagagccatcctccagttttacccgaaatacgtggaactcatcaaccaggctgcccggctcaatggtgagtccctgctgccaacatcactggcacttgggtcccttcattttcctcaaagagg

SEQ ID NO: 11 - DNA, 481 bp, Artificial Sequence
  Synthetic
aaaaaaaaaaaaaaaggagaggagagagactcaagcacgcccctcacaggactgctgaggccctgcaggtgtctgcagcatgtgccccaggccggggactctgtaagccactgctggagaccactcccatcctttctcccatttctctagacctgctgcctatacagtcacttttatgtggtttcgccaattttattccagctctgaaattctctgagctccccttacaagcagaggtgagctaagggctggagctcaaggcattcaaacccctaccagatctgacgaatgtgatggccacatcccggaaatatgaagacctgttatgggcatgggagggctggcgagacaaggcggggagagccatcctccagttttacccgaaatacgtggaactcatcaaccaggctgcccggctcaatggtgagtccctgctgccaacatcactggcacttgggtcccttcattttcctcaaagagg

SEQ ID NO: 12 - DNA, 122 bp, Artificial Sequence
  Synthetic
gagctcccgattgcctcgcgtaactcttccctcttttcctctaatcagacagccgagctcagctccggaacttgtcatctccaacgacaaaaggagcttttgccactgactcggccgtgtcc

SEQ ID NO: 13 - DNA, 122 bp, Artificial Sequence
  Synthetic
agaaaagggaagatgcttgagctgatcccctaggtatagcttctgaaggtctggcttcttagctagatttgctgacctagactccatgctgctcatcctacccccttgcccatgtcctccct

SEQ ID NO: 14 - DNA, 122 bp, Artificial Sequence
  Synthetic
tgcccaggaggggcatacccatctccccttcccttgggtgggcctaactgccctcccttaacgctggaccttcagtaacatcgacattcctctctttgcccagggaaggagtgggcaagccc

SEQ ID NO: 15 - DNA, 122 bp, Artificial Sequence
  Synthetic
agaccacataatctacattaatctatcttcaagtttgcaaattatttcttctgccatctcaccatttgaaactttctagtagattttttacttcagttattgtgcttttcaactctagaatt

SEQ ID NO: 16 - DNA, 122 bp, Artificial Sequence
  Synthetic
ataaccttccaggccccaggacaactttggtggctgggccccagcagcaaccttgtgtctagttacacgagtcgccagtttcaagactgccaaagcagcactcattttctccttcctatttc

SEQ ID NO: 17 - DNA, 122 bp, Artificial Sequence
  Synthetic
gatgacagaacaagtccactgagtcccgcaggccaatgcacggcacctaaaggtgcatggagaccaaactcatcctctccccaggaagcccttggcccccaagaagagagacagttcagcag

SEQ ID NO: 18 - DNA, 122 bp, Artificial Sequence
  Synthetic
acacaaaaattgatcagtgttataggccacattaacagaatgaagggaagatgccacataagttatctcaattgaagccagaaaaaacatttgaaaaaattcagtacccttctatgattaaa

SEQ ID NO: 19 - DNA, 122 bp, Artificial Sequence
  Synthetic
ttctgatatagaattatagaattaggcccagagttaaaaacattgggtttattccctaccagatatgcttatattaaaattaaaatgacagccttcttctttcaaatgcattctaaggcctt

SEQ ID NO: 20 - DNA, 122 bp, Artificial Sequence
  Synthetic
cagggtgaaacattgtaggacaatcagggaagtggaacactgacttgatattaaggaatcagtggttatttttcttaggacaatgacattgatattagttttatatttatattttatatacc

SEQ ID NO: 21 - DNA, 122 bp, Artificial Sequence
  Synthetic
gatttccataagtggacagaggaactttgcccctgtctggaagatagagacaaagaacccagtgaaagaggacatccatcattgtagaaggcgactgctggtgggggagatgaagactctgt

SEQ ID NO: 22 - DNA, 122 bp, Artificial Sequence
  Synthetic
acaacctagacccagcctctcaataaacagtgtcaaaggctttagagagcatggtgtcaaagctcccagattctaaggctgtgactcaacccagtgcactgggctgcctggctgtacacagg

SEQ ID NO: 23 - DNA, 122 bp, Artificial Sequence
  Synthetic
tcacaataggaataaactcagatgttttctgtagtctacaaagtttataatccgggccctagcttatcatacctttatctccttccctggccttctgtctgttcctcccagacactgggttt

SEQ ID NO: 24 - DNA, 122 bp, Artificial Sequence
  Synthetic
tttgaggctgtgacttcactatagagacacagtgtgcagaatggattgaggctctcatacagcaaatccaatccaccgttaagattgtcactcgtggccaggcatggtggctcacgcctgta

SEQ ID NO: 25 - DNA, 122 bp, Artificial Sequence
  Synthetic
cccattagtttttgctgctgtcatctcatttgttagcaggcatcaaagtcgcttaaaagcagggtcacaaaaggctacttggcaacgttccacagaggtgtagattaaagatattttagggt

SEQ ID NO: 26 - DNA, 122 bp, Artificial Sequence
  Synthetic
cttaactattacatgctacatgtaagaagcctagttctctgtctcattatgcagatctccaggacttttcaaatctccatgagatatgaatgaccaacagaacagcaattatgaaaaatttt

SEQ ID NO: 27 - DNA, 122 bp, Artificial Sequence
  Synthetic
cgggtccctggggactcggatggcacagagggccccttcctgccaccatcacggctcagaacctcacgttcctggagagtaggggtggggtgctgaggggcagagggaagtgccgcaaaccc

SEQ ID NO: 28 - DNA, 122 bp, Artificial Sequence; identical to SEQ ID NO: 27
  Synthetic

SEQ ID NO: 29 - DNA, 122 bp, Artificial Sequence
  Synthetic
tgccctatctatgccgtaggacactattcagatgcagtgggatgtctagttggtattcccacagggcattcagtgcagcacccctcacctcccccagcagacctggcacatagaagcaaaga

SEQ ID NO: 30 - DNA, 122 bp, Artificial Sequence
  Synthetic
aatgtctcacagctggaagtgggggtgggggccgcctgctccccagagctccccgtgtccagtctttccatctgtctgtcccccctctcttggctttgtccctcactgggcatctgtacccc

SEQ ID NO: 31 - DNA, 122 bp, Artificial Sequence
  Synthetic
ctgggttacatggcaaatgtgtttttatccttctgaggaacagccagactatgtttcaaaattgtctgtcattttacattcccagcagcagtgcccgggggttcctgtttctccacatcccc

SEQ ID NO: 32 - DNA, 122 bp, Artificial Sequence
  Synthetic
tgccctccaggtacttccctccacctcccagtctctgtttctctgtctttgctcctctccagtcttggctctctgtattttttttgtttttttttttggagacaagggtcccgctctgtcac

SEQ ID NO: 33 - DNA, 122 bp, Artificial Sequence
  Synthetic
tacactggaggaagagaagttgtttcctcttttatgtaaaacattactgcagttcttctcaggtgcaatttgcagagcaactctgagtctacataaataaaaagggaaagaggtggtttttc

SEQ ID NO: 34 - DNA, 122 bp, Artificial Sequence
  Synthetic
tgatttgctgagagaaacagagaactggtccctgagtccccgactccacacctccagtacagacccatgaatttatgtgggatgcatcaaggtgtccctcctagaactggaaccaagactgc

SEQ ID NO: 35 - DNA, 122 bp, Artificial Sequence
  Synthetic
ggtcacaggatgggtgacctttttagttttcacagccattgtccaatcaattagtccaggacctcaccaatgttatttgctgtgacaaggttcaatgctgccttttctgatgggtccagtgg

SEQ ID NO: 36 - DNA, 122 bp, Artificial Sequence
  Synthetic
agcaaaaggacagcattagatggaagctggctcaagaggctcagctcttgcccagaggccagtctgccatagataagacccatcaggcctctgcagctaagacctggcccccaaatctacac

SEQ ID NO: 37 - DNA, 25 bp, Artificial seq
  Synthetic
ttagcaccacaagaatacacaacac

SEQ ID NO: 38 - DNA, 25 bp, Artificial Sequence
  Synthetic
agagatattcaacattcatggatag

SEQ ID NO: 39 - DNA, 25 bp, Artificial Sequence
  Synthetic
gatgtcagttcttcccaacttgatg

SEQ ID NO: 40 - DNA, 25 bp, Artificial Sequence
  Synthetic
gttcttcccaacttgatgtatatat

SEQ ID NO: 41 - DNA, 25 bp, Artificial Sequence
  Synthetic
aaatcctacagagttattttgtgga

SEQ ID NO: 42 - DNA, 25 bp, Artificial Sequence
  Synthetic
gaatagccaacgcagtactgaagga

SEQ ID NO: 43 - DNA, 25 bp, Artificial Sequence
  Synthetic
ccagaggactggcactacttaacgt

SEQ ID NO: 44 - DNA, 25 bp, Artificial Sequence
  Synthetic
tggcactacttaacgtcaagactta

SEQ ID NO: 45 - DNA, 25 bp, Artificial Sequence
  Synthetic
tcaagacttaccgtaaagcgacagt

SEQ ID NO: 46 - DNA, 25 bp, Artificial Sequence
  Synthetic
gtaaagcgacagtaatcacgacagt

SEQ ID NO: 47 - DNA, 25 bp, Artificial Sequence
  Synthetic
atagacctctaccaatagttcagtg

SEQ ID NO: 48 - DNA, 25 bp, Artificial Sequence
  Synthetic
cccttgatggtctgggagcctggcc

SEQ ID NO: 49 - DNA, 25 bp, Artificial Sequence
  Synthetic
atgtcctcactttgtgggtcacact

SEQ ID NO: 50 - DNA, 25 bp, Artificial Sequence
  Synthetic
ggtcacactctttacatttctgtaa

SEQ ID NO: 51 - DNA, 25 bp, Artificial Sequence
  Synthetic
gtaaggcaatcttggcacacgtggg

SEQ ID NO: 52 - DNA, 25 bp, Artificial Sequence
  Synthetic
gcacacgtggggcttaccagtggcc

SEQ ID NO: 53 - DNA, 25 bp, Artificial Sequence
  Synthetic
tccttttgaattttgcacagcccta

SEQ ID NO: 54 - DNA, 25 bp, Artificial Sequence
  Synthetic
cagccctagatacaatcccttttga

SEQ ID NO: 55 - DNA, 25 bp, Artificial Sequence
  Synthetic
ggagcactgtggaacgtctgtaaat

SEQ ID NO: 56 - DNA, 25 bp, Artificial Sequence
  Synthetic
ttggtgtacactcaaaacctgtccc

SEQ ID NO: 57 - DNA, 25 bp, Artificial Sequence
  Synthetic
gcagccagtgctctctgtatagggc

SEQ ID NO: 58 - DNA, 25 bp, Artificial Sequence
  Synthetic
tccagtgctcagacctttagactca

SEQ ID NO: 59 - DNA, 25 bp, Artificial Sequence
  Synthetic
gcgtttccaacctcggagaattcca

SEQ ID NO: 60 - DNA, 25 bp, Artificial Sequence
  Synthetic
gtataagcggtcatcgttgcgtcat

SEQ ID NO: 61 - DNA, 25 bp, Artificial Sequence
  Synthetic
gggtgtgggcctggaggaaggtcct

SEQ ID NO: 62 - DNA, 25 bp, Artificial Sequence
  Synthetic
gagagtggcctgagttacttcaccc

SEQ ID NO: 63 - DNA, 25 bp, Artificial Sequence
  Synthetic
cgcgtgctgctggttaatgtcccgc

SEQ ID NO: 64 - DNA, 25 bp, Artificial Sequence
  Synthetic
ggactgatctactttcacattctca

SEQ ID NO: 65 - DNA, 25 bp, Artificial Sequence
  Synthetic
gcattagaggtccccagtaggttcc

SEQ ID NO: 66 - DNA, 25 bp, Artificial Sequence
  Synthetic
cagccgagaagttcctggtctgaat

SEQ ID NO: 67 - DNA, 25 bp, Artificial Sequence
  Synthetic
gtttctgagggtctgctttgtttac

SEQ ID NO: 68 - DNA, 25 bp, Artificial Sequence
  Synthetic
gtttacctttcgtgcggtggattct

SEQ ID NO: 69 - DNA, 25 bp, Artificial Sequence
  Synthetic
tccgtctacctggcgttttgttaga

SEQ ID NO: 70 - DNA, 25 bp, Artificial Sequence
  Synthetic
ggggtgaaacacccacatggcagcc

SEQ ID NO: 71 - DNA, 25 bp, Artificial Sequence
  Synthetic
cacatggcagcctgctagcagcagt

SEQ ID NO: 72 - DNA, 25 bp, Artificial Sequence
  Synthetic
ctggtcttaaagagtccctcacttc

SEQ ID NO: 73 - DNA, 25 bp, Artificial Sequence
  Synthetic
tcagccccaggagctattggtgggt

SEQ ID NO: 74 - DNA, 25 bp, Artificial Sequence
  Synthetic
tttttagttctccttgattctttgt

SEQ ID NO: 75 - DNA, 25 bp, Artificial Sequence
  Synthetic
tatcgtttttaggtttggtatgtgt

SEQ ID NO: 76 - DNA, 25 bp, Artificial Sequence
  Synthetic
atttccatggttcctcaagtttcct

SEQ ID NO: 77 - DNA, 25 bp, Artificial Sequence
  Synthetic
atacatttggttcatgtgcattgtt

SEQ ID NO: 78 - DNA, 25 bp, Artificial Sequence
  Synthetic
tttttgtgctgtgaacattttctgc

SEQ ID NO: 79 - DNA, 25 bp, Artificial Sequence
  Synthetic
gtgtctgtatgtttaagttatcgta

SEQ ID NO: 80 - DNA, 25 bp, Artificial Sequence
  Synthetic
atggctgttttgttatgccaccctg

SEQ ID NO: 81 - DNA, 25 bp, Artificial Sequence
  Synthetic
acctggagacagtggcggcttatta

SEQ ID NO: 82 - DNA, 25 bp, Artificial Sequence
  Synthetic
ggcttattatgaggagcagcaccca

SEQ ID NO: 83 - DNA, 25 bp, Artificial Sequence
  Synthetic
aagagatggattacggtgccgaggc

SEQ ID NO: 84 - DNA, 25 bp, Artificial Sequence
  Synthetic
tacggtgccgaggcaacagatcccc

SEQ ID NO: 85 - DNA, 25 bp, Artificial Sequence
  Synthetic
atcccctgtcccggatgttgaggat

SEQ ID NO: 86 - DNA, 25 bp, Artificial Sequence
  Synthetic
tcccggatgttgaggatcccgcaac

SEQ ID NO: 87 - DNA, 25 bp, Artificial Sequence
  Synthetic
cccgcaaccgaggagcctggggaga

SEQ ID NO: 88 - DNA, 25 bp, Artificial Sequence
  Synthetic
tgagatggttccaggccatgctgca

SEQ ID NO: 89 - DNA, 25 bp, Artificial Sequence
  Synthetic
ctgctctctgtcagagctcttcatg

SEQ ID NO: 90 - DNA, 25 bp, Artificial Sequence
  Synthetic
ctgacaccccagaagtgctctgaac

SEQ ID NO: 91 - DNA, 25 bp, Artificial Sequence
  Synthetic
atgaagatactgacaccacctttgc

SEQ ID NO: 92 - DNA, 25 bp, Artificial Sequence
  Synthetic
cctctgtgaactggtgcagcacctg

SEQ ID NO: 93 - DNA, 25 bp, Artificial Sequence
  Synthetic
acatatcagtttctgcaagccttga

SEQ ID NO: 94 - DNA, 25 bp, Artificial Sequence
  Synthetic
gtgtgtgagtatgttgaccacctgc

SEQ ID NO: 95 - DNA, 25 bp, Artificial Sequence
  Synthetic
gtatgttgaccacctgcatgagcat

SEQ ID NO: 96 - DNA, 25 bp, Artificial Sequence
  Synthetic
gcatgagcatttcaagtatcccgtg

SEQ ID NO: 97 - DNA, 25 bp, Artificial Sequence
  Synthetic
gtatcccgtgatgatccagcgggct

SEQ ID NO: 98 - DNA, 25 bp, Artificial Sequence
  Synthetic
gtaaagaaacaccagtatccagatg

SEQ ID NO: 99 - DNA, 25 bp, Artificial Sequence
  Synthetic
tccttcctgctcaagaaaattaagt

SEQ ID NO: 100 - DNA, 25 bp, Artificial Sequence
  Synthetic
aaatcctaccgatcaagatgagttc

SEQ ID NO: 101 - DNA, 25 bp, Artificial Sequence
  Synthetic
gttcagctagaagtcataccaccct

SEQ ID NO: 102 - DNA, 25 bp, Artificial Sequence
  Synthetic
cataccaccctcaggaatcagctaa

SEQ ID NO: 103 - DNA, 25 bp, Artificial Sequence
  Synthetic
gaacttgtcatctccaacgacaaaa

SEQ ID NO: 104 - DNA, 25 bp, Artificial Sequence
  Synthetic
aaaaggagcttttgccactgactcg

SEQ ID NO: 105 - DNA, 25 bp, Artificial Sequence
  Synthetic
cctccagaacgcaggtgctggcgcc

SEQ ID NO: 106 - DNA, 25 bp, Artificial Sequence
  Synthetic
ggaagccggacggcagggatgggcc

SEQ ID NO: 107 - DNA, 25 bp, Artificial Sequence
  Synthetic
ggtgctcaggagcgaaggactgtga

SEQ ID NO: 108 - DNA, 25 bp, Artificial Sequence
  Synthetic
gtggcctgaagagccagagctagct

SEQ ID NO: 109 - DNA, 25 bp, Artificial Sequence
  Synthetic
ggtcttttcagagcgtggaggtgtg

SEQ ID NO: 110 - DNA, 25 bp, Artificial Sequence
  Synthetic
gaaggagtggctgctctccaaacta

SEQ ID NO: 111 - DNA, 25 bp, Artificial Sequence
  Synthetic
ctgctctccaaactatgccaaggcg

SEQ ID NO: 112 - DNA, 25 bp, Artificial Sequence
  Synthetic
actatgccaaggcggcggcagagct

SEQ ID NO: 113 - DNA, 25 bp, Artificial Sequence
  Synthetic
ttggagaaaggttctgttgccctga

SEQ ID NO: 114 - DNA, 25 bp, Artificial Sequence
  Synthetic
gaaatttttgtcactcccagaggtg

SEQ ID NO: 115 - DNA, 25 bp, Artificial Sequence
  Synthetic
gacaagccatccacgtggggaatca

SEQ ID NO: 116 - DNA, 25 bp, Artificial Sequence
  Synthetic
acagtacagtcagttaagccatggt

SEQ ID NO: 117 - DNA, 25 bp, Artificial Sequence
  Synthetic
taaggttctgatctacaatggccaa

SEQ ID NO: 118 - DNA, 25 bp, Artificial Sequence
  Synthetic
caatggccaactggacatcatcgtg

SEQ ID NO: 119 - DNA, 25 bp, Artificial Sequence
  Synthetic
acagagcactccttgatgggcatgg

SEQ ID NO: 120 - DNA, 25 bp, Artificial Sequence
  Synthetic
gtgaagtggctggttacatccggca

SEQ ID NO: 121 - DNA, 25 bp, Artificial Sequence
  Synthetic
ttacatccggcaagcgggtgactcc

SEQ ID NO: 122 - DNA, 25 bp, Artificial Sequence
  Synthetic
gggtgactcccatcaggtaattatt

SEQ ID NO: 123 - DNA, 25 bp, Artificial Sequence
  Synthetic
gacatattttaccctatgaccagcc

SEQ ID NO: 124 - DNA, 25 bp, Artificial Sequence
  Synthetic
tatgttggataaactaccttcccga

SEQ ID NO: 125 - DNA, 25 bp, Artificial Sequence
  Synthetic
gaagacaaatcaactgcaacgcatc

SEQ ID NO: 126 - DNA, 25 bp, Artificial Sequence
  Synthetic
aacgcatcattcggacaggccgtac

SEQ ID NO: 127 - DNA, 25 bp, Artificial Sequence
  Synthetic
ggccgtacaggtcactggttgaacc

SEQ ID NO: 128 - DNA, 25 bp, Artificial Sequence
  Synthetic
atccccaaggcttcaaccagggtct

SEQ ID NO: 129 - DNA, 25 bp, Artificial Sequence
  Synthetic
ggttcgttccaccagtcataaacca

SEQ ID NO: 130 - DNA, 25 bp, Artificial Sequence
  Synthetic
tatctcctggcactcgcaagattga

SEQ ID NO: 131 - DNA, 25 bp, Artificial Sequence
  Synthetic
ggacgaccacacaatgtgcaaccca

SEQ ID NO: 132 - DNA, 25 bp, Artificial Sequence
  Synthetic
aatgtgcaacccaactggatcaccc

SEQ ID NO: 133 - DNA, 25 bp, Artificial Sequence
  Synthetic
ggatcacccttggaaaccaactgga

SEQ ID NO: 134 - DNA, 25 bp, Artificial Sequence
  Synthetic
tggatgggatccacctactagaccc

SEQ ID NO: 135 - DNA, 25 bp, Artificial Sequence
  Synthetic
gccatggctctgtaagctaaacctg

SEQ ID NO: 136 - DNA, 25 bp, Artificial Sequence
  Synthetic
tgcatagatgtacctatcctgcacc

SEQ ID NO: 137 - DNA, 25 bp, Artificial Sequence
  Synthetic
gtacctatcctgcacccaaaaaggt

SEQ ID NO: 138 - DNA, 25 bp, Artificial Sequence
  Synthetic
atcatgtagttatactgggcagcaa

SEQ ID NO: 139 - DNA, 25 bp, Artificial Sequence
  Synthetic
gggcatgaggctgattactcaatgg

SEQ ID NO: 140 - DNA, 25 bp, Artificial Sequence
  Synthetic
tacaggtaataaacatccccaaggt

SEQ ID NO: 141 - DNA, 25 bp, Artificial Sequence
  Synthetic
gtggctggccatacacataggcatc

SEQ ID NO: 142 - DNA, 25 bp, Artificial Sequence
  Synthetic
atcagtttaacaaccatcagacctc

SEQ ID NO: 143 - DNA, 25 bp, Artificial Sequence
  Synthetic
agacctcagctgtacaataacaggt

SEQ ID NO: 144 - DNA, 25 bp, Artificial Sequence
  Synthetic
gttctgcagcatttagacatttgtc

SEQ ID NO: 145 - DNA, 25 bp, Artificial Sequence
  Synthetic
ttagctttgacaaccatactgtaac

SEQ ID NO: 146 - DNA, 25 bp, Artificial Sequence
  Synthetic
gtaacattaaacctagcattccaca

SEQ ID NO: 147 - DNA, 25 bp, Artificial Sequence
  Synthetic
aaacctgtgcttgatctgacatttg

SEQ ID NO: 148 - DNA, 25 bp, Artificial Sequence
  Synthetic
gcatgattcaccaagcagtactaca

SEQ ID NO: 149 - DNA, 25 bp, Artificial Sequence
  Synthetic
gttcacatgttccaactttcaggtt

SEQ ID NO: 150 - DNA, 25 bp, Artificial Sequence
  Synthetic
gtaaccacctacaatagctttcaat

SEQ ID NO: 151 - DNA, 25 bp, Artificial Sequence
  Synthetic
ttcaatttcaattaactcccttggc

SEQ ID NO: 152 - DNA, 25 bp, Artificial Sequence
  Synthetic
aactcccttggctataagcatctaa

SEQ ID NO: 153 - DNA, 25 bp, Artificial Sequence
  Synthetic
gcatctaaactcatcttctttcaat

SEQ ID NO: 154 - DNA, 25 bp, Artificial Sequence
  Synthetic
gctatctcctaattacttggtggct

SEQ ID NO: 155 - DNA, 25 bp, Artificial Sequence
  Synthetic
gaacccttggatttatgtgaggtca

SEQ ID NO: 156 - DNA, 25 bp, Artificial Sequence
  Synthetic
ggtcaaaaccaaactcttattctca

SEQ ID NO: 157 - DNA, 25 bp, Artificial Sequence
  Synthetic
atgtatttcataattctcccataat

SEQ ID NO: 158 - DNA, 25 bp, Artificial Sequence
  Synthetic
ctccacctctgggaagctgagcatg

SEQ ID NO: 159 - DNA, 25 bp, Artificial Sequence
  Synthetic
gagcatgtggtcctggaaatccctt

SEQ ID NO: 160 - DNA, 25 bp, Artificial Sequence
  Synthetic
gaaatcccttattgagggcccagac

SEQ ID NO: 161 - DNA, 25 bp, Artificial Sequence
  Synthetic
cagacagggcatccccaagcagaaa

SEQ ID NO: 162 - DNA, 25 bp, Artificial Sequence
  Synthetic
gcatccccaagcagaaaggcaacca

SEQ ID NO: 163 - DNA, 25 bp, Artificial Sequence
  Synthetic
ggcaaccatggcaggtgggctagcc

SEQ ID NO: 164 - DNA, 25 bp, Artificial Sequence
  Synthetic
aacctgtctcccagggagcagggga

SEQ ID NO: 165 - DNA, 25 bp, Artificial Sequence
  Synthetic
ggcccatccatcttatgaggatccc

SEQ ID NO: 166 - DNA, 25 bp, Artificial Sequence
  Synthetic
ggctggctatgggagtctgagtgtg

SEQ ID NO: 167 - DNA, 25 bp, Artificial Sequence
  Synthetic
ggagtctgagtgtgcacaagcagtg

SEQ ID NO: 168 - DNA, 25 bp, Artificial Sequence
  Synthetic
gtgaaagaggatccagccctgagca

SEQ ID NO: 169 - DNA, 25 bp, Artificial Sequence
  Synthetic
gaactgccttactagatttctattt

SEQ ID NO: 170 - DNA, 25 bp, Artificial Sequence
  Synthetic
atttgtagctctcattcattgtttt

SEQ ID NO: 171 - DNA, 25 bp, Artificial Sequence
  Synthetic
cttctctagcccaaacagcgacatg

SEQ ID NO: 172 - DNA, 25 bp, Artificial Sequence
  Synthetic
agtccccttcttcagagtcaataga

SEQ ID NO: 173 - DNA, 25 bp, Artificial Sequence
  Synthetic
aagacctgttcactagcattttcaa

SEQ ID NO: 174 - DNA, 25 bp, Artificial Sequence
  Synthetic
aagggggttctaaagcattcaagtg

SEQ ID NO: 175 - DNA, 25 bp, Artificial Sequence
  Synthetic
aaatgacttcttaattcctgccttt

SEQ ID NO: 176 - DNA, 25 bp, Artificial Sequence
  Synthetic
aattcctgcctttagtgtcaacttt

SEQ ID NO: 177 - DNA, 25 bp, Artificial Sequence
  Synthetic
tacaggtttcaattgtggcattagg

SEQ ID NO: 178 - DNA, 25 bp, Artificial Sequence
  Synthetic
gactacatgaaattgtgtgccccta

SEQ ID NO: 179 - DNA, 25 bp, Artificial Sequence
  Synthetic
aatcagctatagcatctttctagaa

SEQ ID NO: 180 - DNA, 25 bp, Artificial Sequence
  Synthetic
gttgatgccaaaatacccacggggt

SEQ ID NO: 181 - DNA, 25 bp, Artificial Sequence
  Synthetic
taccagccatggggtttgcttgctt

SEQ ID NO: 182 - DNA, 25 bp, Artificial Sequence
  Synthetic
cagaggtgattacaggcctgggttt

SEQ ID NO: 183 - DNA, 25 bp, Artificial Sequence
  Synthetic
gcctgggtttgactgtgcttaccaa

SEQ ID NO: 184 - DNA, 25 bp, Artificial Sequence
  Synthetic
tctttatgagcctcgatgttccctg

SEQ ID NO: 185 - DNA, 25 bp, Artificial Sequence
  Synthetic
aggccttctctcatgatctaagtct

SEQ ID NO: 186 - DNA, 25 bp, Artificial Sequence
  Synthetic
aagtcttggactggtggcatcatgt

SEQ ID NO: 187 - DNA, 25 bp, Artificial Sequence
  Synthetic
ggtggcatcatgtaactgctaacct

SEQ ID NO: 188 - DNA, 25 bp, Artificial Sequence
  Synthetic
tctggaatgcaggtctgtcggctgg

SEQ ID NO: 189 - DNA, 25 bp, Artificial Sequence
  Synthetic
tgctcctgcctgattcaactgtagc

SEQ ID NO: 190 - DNA, 25 bp, Artificial Sequence
  Synthetic
gtccatgagactttctgactaggaa

SEQ ID NO: 191 - DNA, 25 bp, Artificial Sequence
  Synthetic
atccgacttgaatattcctggactt

SEQ ID NO: 192 - DNA, 25 bp, Artificial Sequence
  Synthetic
gccaagggggtgactggaagttgtg

SEQ ID NO: 193 - DNA, 25 bp, Artificial Sequence
  Synthetic
ggaagaccagaattcccttgaattg

SEQ ID NO: 194 - DNA, 25 bp, Artificial Sequence
  Synthetic
aaagatcaccttgtattctctttac

SEQ ID NO: 195 - DNA, 25 bp, Artificial Sequence
  Synthetic
gatggtgcttggtgagtcttggttc

SEQ ID NO: 196 - DNA, 25 bp, Artificial Sequence
  Synthetic
aaactgctgcatactttgacaagga

SEQ ID NO: 197 - DNA, 25 bp, Artificial Sequence
  Synthetic
aatctatatttgtcttccgatcaac

SEQ ID NO: 198 - DNA, 25 bp, Artificial Sequence
  Synthetic
atacctggtttacttctttagcatt

SEQ ID NO: 199 - DNA, 25 bp, Artificial Sequence
  Synthetic
cagacagtctgttatgcactgtggt

SEQ ID NO: 200 - DNA, 25 bp, Artificial Sequence
  Synthetic
ggtttattcccaagtatgccttaag

SEQ ID NO: 201 - DNA, 25 bp, Artificial Sequence
  Synthetic
ttttctatatagttccttgccttaa

SEQ ID NO: 202 - DNA, 25 bp, Artificial Sequence
  Synthetic
ggaagcttggtgcagacgatgtaat

SEQ ID NO: 203 - DNA, 25 bp, Artificial Sequence
  Synthetic
ggcggatccactgaaacatgggctc

SEQ ID NO: 204 - DNA, 25 bp, Artificial Sequence
  Synthetic
acatgggctccagattttctcaaga

SEQ ID NO: 205 - DNA, 25 bp, Artificial Sequence
  Synthetic
gaaatggtcaggagccacctatgtg

SEQ ID NO: 206 - DNA, 25 bp, Artificial Sequence
  Synthetic
tatgtgactttggtgactcctttcc

SEQ ID NO: 207 - DNA, 25 bp, Artificial Sequence
  Synthetic
ttcctcctgaacatggaccgattgg

SEQ ID NO: 208 - DNA, 25 bp, Artificial Sequence
  Synthetic
ggcatgttgcagacaggagtcactg

SEQ ID NO: 209 - DNA, 25 bp, Artificial Sequence
  Synthetic
gaaaggagtccattatcgctgggca

SEQ ID NO: 210 - DNA, 25 bp, Artificial Sequence
  Synthetic
tatcgctgggcatttttcatggcca

SEQ ID NO: 211 - DNA, 25 bp, Artificial Sequence
  Synthetic
ggccagtggcccatgtttagatgac

SEQ ID NO: 212 - DNA, 25 bp, Artificial Sequence
  Synthetic
ggaaagatccggccagttattgaac

SEQ ID NO: 213 - DNA, 25 bp, Artificial Sequence
  Synthetic
ccttctgtctctttgtttctgagct

SEQ ID NO: 214 - DNA, 25 bp, Artificial Sequence
  Synthetic
cttctgtctctttgtttctgagctt

SEQ ID NO: 215 - DNA, 25 bp, Artificial Sequence
  Synthetic
ttctgtctctttgtttctgagcttt

SEQ ID NO: 216 - DNA, 25 bp, Artificial Sequence
  Synthetic
tctgtctctttgtttctgagctttc

SEQ ID NO: 217 - DNA, 25 bp, Artificial Sequence
  Synthetic
ctgtctctttgtttctgagctttcc

SEQ ID NO: 218 - DNA, 25 bp, Artificial Sequence
  Synthetic
tgtctctttgtttctgagctttcct

SEQ ID NO: 219 - DNA, 25 bp, Artificial Sequence
  Synthetic
tctctttgtttctgagctttcctgt

SEQ ID NO: 220 - DNA, 25 bp, Artificial Sequence
  Synthetic
gaagctccgaccgacatcacggagc

SEQ ID NO: 221 - DNA, 25 bp, Artificial Sequence
  Synthetic
agctccgaccgacatcacggagcag

SEQ ID NO: 222 - DNA, 25 bp, Artificial Sequence
  Synthetic
ctccgaccgacatcacggagcagcc

SEQ ID NO: 223 - DNA, 25 bp, Artificial Sequence
  Synthetic
tcacggagcagccttcaagcattcc

SEQ ID NO: 224 - DNA, 25 bp, Artificial Sequence
  Synthetic
gggatgtgtattagccccggaggac

SEQ ID NO: 225 - DNA, 25 bp, Artificial Sequence
  Synthetic
tagccccggaggacgtgatgtgaga

SEQ ID NO: 226 - DNA, 25 bp, Artificial Sequence
  Synthetic
tgatgtgagacccgcttgtgagtcc

SEQ ID NO: 227 - DNA, 25 bp, Artificial Sequence
  Synthetic
cactcgttccccattggcaagatac

SEQ ID NO: 228 - DNA, 25 bp, Artificial Sequence
  Synthetic
tacatggagagcaccctgaggacct

SEQ ID NO: 229 - DNA, 25 bp, Artificial Sequence
  Synthetic
gtccctgaatcaccgactggaggag

SEQ ID NO: 230 - DNA, 25 bp, Artificial Sequence
  Synthetic
gagttacctacaagagccttcatcc

SEQ ID NO: 231 - DNA, 25 bp, Artificial Sequence
  Synthetic
ccaggagcatccacactgcaatgat

SEQ ID NO: 232 - DNA, 25 bp, Artificial Sequence
  Synthetic
aggaatgaggtctgaactccactga

SEQ ID NO: 233 - DNA, 25 bp, Artificial Sequence
  Synthetic
tgaactccactgaattaaaccactg

SEQ ID NO: 234 - DNA, 25 bp, Artificial Sequence
  Synthetic
gcagtgcaaagagttcctttatcct

SEQ ID NO: 235 - DNA, 25 bp, Artificial Sequence
  Synthetic
ccactcatctactcattcttcgagt

SEQ ID NO: 236 - DNA, 25 bp, Artificial Sequence
  Synthetic
gagtctacacttattgaatgcctgc

SEQ ID NO: 237 - DNA, 25 bp, Artificial Sequence
  Synthetic
gatctctctctcaataggtttctta

SEQ ID NO: 238 - DNA, 25 bp, Artificial Sequence
  Synthetic
ttgtgacgcttgttgcagtttacca

SEQ ID NO: 239 - DNA, 25 bp, Artificial Sequence
  Synthetic
aatgtttccattccgttgttgtagt

SEQ ID NO: 240 - DNA, 25 bp, Artificial Sequence
  Synthetic
taagctgattaccccactgtgggaa

SEQ ID NO: 241 - DNA, 25 bp, Artificial Sequence
  Synthetic
ggattcctactttgttggactctct

SEQ ID NO: 242 - DNA, 25 bp, Artificial Sequence
  Synthetic
ttggactctctttcctgattttaac

SEQ ID NO: 243 - DNA, 25 bp, Artificial Sequence
  Synthetic
tttaacaatttaccatcccattctc

SEQ ID NO: 244 - DNA, 25 bp, Artificial Sequence
  Synthetic
gtgattgtatgctggctacactgct

SEQ ID NO: 245 - DNA, 25 bp, Artificial Sequence
  Synthetic
gctacactgcttttagaatgctctt

SEQ ID NO: 246 - DNA, 25 bp, Artificial Sequence
  Synthetic
atctgttatcgctgaagtttctctt

SEQ ID NO: 247 - DNA, 25 bp, Artificial Sequence
  Synthetic
caggccttggacctagttgatcgac

SEQ ID NO: 248 - DNA, 25 bp, Artificial Sequence
  Synthetic
ttgatcgacagtccatcaccttaat

SEQ ID NO: 249 - DNA, 25 bp, Artificial Sequence
  Synthetic
caccttaatctcatcacccagtgga

SEQ ID NO: 250 - DNA, 25 bp, Artificial Sequence
  Synthetic
gaaggcgtgtttaccaggtccttgg

SEQ ID NO: 251 - DNA, 25 bp, Artificial Sequence
  Synthetic
ttggcttcttgtcattactgttcat

SEQ ID NO: 252 - DNA, 25 bp, Artificial Sequence
  Synthetic
tactgttcatgtcctgcatttgcat

SEQ ID NO: 253 - DNA, 25 bp, Artificial Sequence
  Synthetic
gcatttgcattctcagtgctacgga

SEQ ID NO: 254 - DNA, 25 bp, Artificial Sequence
  Synthetic
aagcatctcttggcagtttacctga

SEQ ID NO: 255 - DNA, 25 bp, Artificial Sequence
  Synthetic
gagaagccctgtacagtcttgtcaa

SEQ ID NO: 256 - DNA, 25 bp, Artificial Sequence
  Synthetic
agccagtctctgagacgcttcggta

SEQ ID NO: 257 - DNA, 25 bp, Artificial Sequence
  Synthetic
ccagagttttttacttcctcacgcg

SEQ ID NO: 258 - DNA, 25 bp, Artificial Sequence
  Synthetic
tcctcacgcgattgtaggttcctct

SEQ ID NO: 259 - DNA, 25 bp, Artificial Sequence
  Synthetic
gagaccgcttaatcagcagcttgac

SEQ ID NO: 260 - DNA, 25 bp, Artificial Sequence
  Synthetic
aacagtttaatcactcccaagtcct

SEQ ID NO: 261 - DNA, 25 bp, Artificial Sequence
  Synthetic
ctgggcaacagatgaccttcaagtc

SEQ ID NO: 262 - DNA, 25 bp, Artificial Sequence
  Synthetic
cctccgctctccggggagatgggaa

SEQ ID NO: 263 - DNA, 25 bp, Artificial Sequence
  Synthetic
gggagatgggaaggctctcctctcg

SEQ ID NO: 264 - DNA, 25 bp, Artificial Sequence
  Synthetic
gaggccccacaagtgtttggctaag

SEQ ID NO: 265 - DNA, 25 bp, Artificial Sequence
  Synthetic
ttggctaagcacaggctctcgggaa

SEQ ID NO: 266 - DNA, 25 bp, Artificial Sequence
  Synthetic
caggctctcgggaatttaacacttt

SEQ ID NO: 267 - DNA, 25 bp, Artificial Sequence
  Synthetic
gggaaggaataggccctttgtgctg

SEQ ID NO: 268 - DNA, 25 bp, Artificial Sequence
  Synthetic
caaagaatggctggcagcgctgcca

SEQ ID NO: 269 - DNA, 25 bp, Artificial Sequence
  Synthetic
tcagggatggctcctaggtggctga

SEQ ID NO: 270 - DNA, 25 bp, Artificial Sequence
  Synthetic
cctgtcgtctgtaactctagtgttc

SEQ ID NO: 271 - DNA, 25 bp, Artificial Sequence
  Synthetic
aactctagtgttcgacattcgccgt

SEQ ID NO: 272 - DNA, 25 bp, Artificial Sequence
  Synthetic
gacattcgccgtgatacagtggtgt

SEQ ID NO: 273 - DNA, 25 bp, Artificial Sequence
  Synthetic
tccgcgtggacgcctcaagtggatt

SEQ ID NO: 274 - DNA, 25 bp, Artificial Sequence
  Synthetic
caagtggattaatttctggaagcct

SEQ ID NO: 275 - DNA, 25 bp, Artificial Sequence
  Synthetic
tggaagcctcaatctgtatgtttga

SEQ ID NO: 276 - DNA, 25 bp, Artificial Sequence
  Synthetic
aatcatttacttgtagcgaactgtt

SEQ ID NO: 277 - DNA, 25 bp, Artificial Sequence
  Synthetic
ttttttacactatagcatttatgca

SEQ ID NO: 278 - DNA, 25 bp, Artificial Sequence
  Synthetic
tggtttacagaattcatggagttat

SEQ ID NO: 279 - DNA, 25 bp, Artificial Sequence
  Synthetic
tatattcactcctgccaaggactcc

SEQ ID NO: 280 - DNA, 25 bp, Artificial Sequence
  Synthetic
agagcaaggaagcctcgttctcttt

SEQ ID NO: 281 - DNA, 25 bp, Artificial Sequence
  Synthetic
ttgatttaggctacggcctcactct

SEQ ID NO: 282 - DNA, 25 bp, Artificial Sequence
  Synthetic
actctctatggccaccctaagagga

SEQ ID NO: 283 - DNA, 25 bp, Artificial Sequence
  Artificial Sequence
ttcacctcattacctccagagggct

SEQ ID NO: 284 - DNA, 25 bp, Artificial Sequence
  Synthetic
ctgggcagggccaagtgcctcatag

SEQ ID NO: 285 - DNA, 25 bp, Artificial Sequence
  Synthetic
gcctcataggactcatgttctctcc

SEQ ID NO: 286 - DNA, 25 bp, Artificial Sequence
  Synthetic
tgggcagggtacttgccctttgtcc

SEQ ID NO: 287 - DNA, 25 bp, Artificial Sequence
  Synthetic
cacctaggacctttcctggacatga

SEQ ID NO: 288 - DNA, 25 bp, Artificial Sequence
  Synthetic
gacatgagtttccttcactatcata

SEQ ID NO: 289 - DNA, 25 bp, Artificial Sequence
  Synthetic
tcatagtcatgagcctcctacttct

SEQ ID NO: 290 - DNA, 25 bp, Artificial Sequence
  Synthetic
ggtcatcgaatctgcatgcatccct

SEQ ID NO: 291 - DNA, 25 bp, Artificial Sequence
  Synthetic
atgcatccctcatacatctggagac

SEQ ID NO: 292 - DNA, 25 bp, Artificial Sequence
  Synthetic
gaaggttccagagttactgactgag

SEQ ID NO: 293 - DNA, 25 bp, Artificial Sequence
  Synthetic
tgactgagatttctgagcttttttc

SEQ ID NO: 294 - DNA, 25 bp, Artificial Sequence
  Synthetic
ctcccaaacacatcgctccttgggg

SEQ ID NO: 295 - DNA, 25 bp, Artificial Sequence
  Synthetic
atcgctccttggggttacactaggt

SEQ ID NO: 296 - DNA, 25 bp, Artificial Sequence
  Synthetic
actaggtttgtttccatctggcttg

SEQ ID NO: 297 - DNA, 25 bp, Artificial Sequence
  Synthetic
ggcttgaggctatttgcaggcgaga

SEQ ID NO: 298 - DNA, 25 bp, Artificial Sequence
  Synthetic
gcaggcgagagtgcagagtctgtaa

SEQ ID NO: 299 - DNA, 25 bp, Artificial Sequence
  Synthetic
ctgtaatgaacctcccagattctct

SEQ ID NO: 300 - DNA, 25 bp, Artificial Sequence
  Synthetic
cagattctctgacgaaggggtcccc

SEQ ID NO: 301 - DNA, 25 bp, Artificial Sequence
  Synthetic
gtggaagaagctcagcttgcccaag

SEQ ID NO: 302 - DNA, 25 bp, Artificial Sequence
  Synthetic
gaagctcagcttgcccaagaagtca

SEQ ID NO: 303 - DNA, 25 bp, Artificial Sequence
  Synthetic
ggaatatcaagaatatcgccaaaca

SEQ ID NO: 304 - DNA, 25 bp, Artificial Sequence
  Synthetic
gggaaggagcctatacacacttcta

SEQ ID NO: 305 - DNA, 25 bp, Artificial Sequence
  Synthetic
gagcctatacacacttctagaggag

SEQ ID NO: 306 - DNA, 25 bp, Artificial Sequence
  Synthetic
ggagatacgggacctagctctcctg

SEQ ID NO: 307 - DNA, 25 bp, Artificial Sequence
  Synthetic
atttaatgtgtgtcactcagtgctc

SEQ ID NO: 308 - DNA, 25 bp, Artificial Sequence
  Synthetic
tgtcactcagtgctctagtcgatca

SEQ ID NO: 309 - DNA, 25 bp, Artificial Sequence
  Synthetic
gtgctctagtcgatcaggactgggt

SEQ ID NO: 310 - DNA, 25 bp, Artificial Sequence
  Synthetic
aggactgggtagctatttcgcatat

SEQ ID NO: 311 - DNA, 25 bp, Artificial Sequence
  Synthetic
gggtagctatttcgcatatatgtaa

SEQ ID NO: 312 - DNA, 25 bp, Artificial Sequence
  Synthetic
accagctacagagacgtttcttccc

SEQ ID NO: 313 - DNA, 25 bp, Artificial Sequence
  Synthetic
aaatcaaactatcttcttctcctta

SEQ ID NO: 314 - DNA, 25 bp, Artificial Sequence
  Synthetic
tcttctccttagccgttcaaatagc

SEQ ID NO: 315 - DNA, 25 bp, Artificial Sequence
  Synthetic
gaaatacacaggcctcttttcgttt

SEQ ID NO: 316 - DNA, 25 bp, Artificial Sequence
  Synthetic
ggcacatcatgcctaggttgctttg

SEQ ID NO: 317 - DNA, 25 bp, Artificial Sequence
  Synthetic
atcacttcctcctaaagcagtctta

SEQ ID NO: 318 - DNA, 25 bp, Artificial Sequence
  Synthetic
gcatagtcatagtctgtgatctcag

SEQ ID NO: 319 - DNA, 25 bp, Artificial Sequence
  Synthetic
tgcttccttctagaacatctgagtt

SEQ ID NO: 320 - DNA, 25 bp, Artificial Sequence
  Synthetic
gacatcactggccttcaacaggtgt

SEQ ID NO: 321 - DNA, 25 bp, Artificial Sequence
  Synthetic
tggatggccacagatcatccacctg

SEQ ID NO: 322 - DNA, 25 bp, Artificial Sequence
  Synthetic
atccacctgccaaacagttaaccct

SEQ ID NO: 323 - DNA, 25 bp, Artificial Sequence
  Synthetic
cagacaccacaacatcctagatgga

SEQ ID NO: 324 - DNA, 25 bp, Artificial Sequence
  Synthetic
cacacctggccgaaataataatatt

SEQ ID NO: 325 - DNA, 25 bp, Artificial Sequence
  Synthetic
attaaatctcttgttcctgtatctc

SEQ ID NO: 326 - DNA, 25 bp, Artificial Sequence
  Synthetic
gttcctgtatctctacatgagctgc

SEQ ID NO: 327 - DNA, 25 bp, Artificial Sequence
  Synthetic
gtatctctacatgagctgcactaat

SEQ ID NO: 328 - DNA, 25 bp, Artificial Sequence
  Synthetic
gagctgcactaataatttgaatctg

SEQ ID NO: 329 - DNA, 25 bp, Artificial Sequence
  Synthetic
aagtgaaacatttaccgttctcata

SEQ ID NO: 330 - DNA, 25 bp, Artificial Sequence
  Synthetic
taccgttctcatatactgataccca

SEQ ID NO: 331 - DNA, 25 bp, Artificial Sequence
  Synthetic
tactgatacccaactaccatgaaat

SEQ ID NO: 332 - DNA, 25 bp, Artificial Sequence
  Synthetic
tttttactcttaatctagtaggtct

SEQ ID NO: 333 - DNA, 25 bp, Artificial Sequence
  Synthetic
gtcactgtctgggaatttaagtggc

SEQ ID NO: 334 - DNA, 25 bp, Artificial Sequence
  Synthetic
gagtttttaagtcctgatctgttct

SEQ ID NO: 335 - DNA, 25 bp, Artificial Sequence
  Synthetic
gtcctgatctgttctaaggtgcctt

SEQ ID NO: 336 - DNA, 25 bp, Artificial Sequence
  Synthetic
gtgattctgaagttcttaatttgca

SEQ ID NO: 337 - DNA, 25 bp, Artificial Sequence
  Synthetic
ggaaatcaggcacaaattgaccaat

SEQ ID NO: 338 - DNA, 25 bp, Artificial Sequence
  Synthetic
attgaccaattctcatgccatttgc

SEQ ID NO: 339 - DNA, 25 bp, Artificial Sequence
  Synthetic
ggatgatgaaacctggctaactaaa

SEQ ID NO: 340 - DNA, 25 bp, Artificial Sequence
  Synthetic
tattaacttgtctccctagaagctg

SEQ ID NO: 341 - DNA, 25 bp, Artificial Sequence
  Synthetic
gaagctgagatttttcgccttaaat

SEQ ID NO: 342 - DNA, 25 bp, Artificial Sequence
  Synthetic
taagtaagcagttctaagtcatgta

SEQ ID NO: 343 - DNA, 25 bp, Artificial Sequence
  Synthetic
caatgcaattgtctgtttcctgaaa

SEQ ID NO: 344 - DNA, 25 bp, Artificial Sequence
  Synthetic
tttgctctcttttactgggattatt

SEQ ID NO: 345 - DNA, 25 bp, Artificial Sequence
  Synthetic
gacagaggggagcggggacaagttt

SEQ ID NO: 346 - DNA, 25 bp, Artificial Sequence
  Synthetic
ttttaagtctaagcctcctgggtgg

SEQ ID NO: 347 - DNA, 25 bp, Artificial Sequence
  Synthetic
gtttcaacatatgctccagtcatgg

SEQ ID NO: 348 - DNA, 25 bp, Artificial Sequence
  Synthetic
gctccagtcatggcagactttggcc

SEQ ID NO: 349 - DNA, 25 bp, Artificial Sequence
  Synthetic
cagcgccctttttcagagtgaactg

SEQ ID NO: 350 - DNA, 25 bp, Artificial Sequence
  Synthetic
tatctgccagtgctagttagcaaac

SEQ ID NO: 351 - DNA, 25 bp, Artificial Sequence
  Synthetic
gcccaaggaatttgaaaccgttgag

SEQ ID NO: 352 - DNA, 25 bp, Artificial Sequence
  Synthetic
actttccgtttttgctacactgatt

SEQ ID NO: 353 - DNA, 25 bp, Artificial Sequence
  Synthetic
gctacactgatttatgttgtgctgg

SEQ ID NO: 354 - DNA, 25 bp, Artificial Sequence
  Synthetic
tgtacaagcctttgaccagacctta

SEQ ID NO: 355 - DNA, 25 bp, Artificial Sequence
  Synthetic
gtgacttgcaaaagcatttttacct

(19) United States
(12) Patent Application Publication
(10) Pub. No.: US 2011/0195412 A1
(43) Pub. Date: Aug. 11, 2011

(54) Predictive Biomarkers for Response to Exercise

(76) Inventors: James Timmons, Camden (GB); Steen Knudsen, Birkeroed (DK); Tuomo Rankinen, Baton Rouge, LA (US); Carl Johan Sundberg, Oesterskaer (SE); Claude Bouchard, Baton Rouge, LA (US)

(21) Appl. No.: 13/061,822

(22) PCT Filed: Sep. 4, 2009
(86) PCT No.: PCT/US09/56057

§ 371 Date: Apr. 29, 2011

(30) Foreign Application Priority Data
Sep. 5, 2008 (DK) PA 2008 01240

(51) Int. Cl. C12Q 1/68; C07H 21/00

(52) U.S. Cl. 435/6.11; 435/6.1; 536/23.2

(57) ABSTRACT

A set of biomarkers have been identified that allows one to predict subjects who will respond to an exercise regime in term of cardiorespiratory fitness as assessed by maximal oxygen uptake. These predictions may be used, for example, to predict risk of cardiovascular disease, to design a more effective program for cardiac rehabilitation, to predict capacity for athletic performance or physically demanding occupation, and to predict ability to maintain functional capacity with aging using exercise.

The development of this invention was partially funded by the United States Government under a grant from the National Institutes of Health, grant nos. HL-45670, HL-47323, HL-47317, HL-47327, HL47321. The United States Government has certain rights in this invention.

### TECHNICAL FIELD

The invention features biomarkers predictive of subjects who will respond to an exercise regime in term of cardiorespiratory fitness as assessed by maximal oxygen uptake, referred to herein as VO2max. In a given subject, these biomarkers can be used to predict the level of gains in VO2max which is relevant to a number of fields including fitness programs for children, adults and seniors, training programs for athletes, selection plans designed to identify recruits with the potential to perform in a number of physically demanding jobs such as those in police forces, firefighter crews and military services, preventive medicine programs with an exercise component aimed at reducing the risk of developing cardiovascular disease and Type 2 diabetes mellitus, and success of therapy programs designed to improve physical working capacity. This information can be used in diagnosis, prognosis and selection of candidates for prevention, treatment and rehabilitation programs as well as in other areas of personalized medicine.

### BACKGROUND ART

Many clinical interventions whether they be life-style modification or pharmacological therapy yield highly variable benefits in the population as a whole. It is critical to develop testing to predict outcome more accurately for the individual, not the group. For example, low aerobic capacity is a clinically established biomarker and risk factor for developing cardiovascular and metabolic disease, and premature death. It is possible to increase aerobic capacity with regular exercise therapy thus reducing disease burden and improving quality of life and decreasing the risk of premature death. However, at much as 15 to 20% of people (also shown in other mammals, e.g., rodents) do not respond to supervised exercise (little or no improvement in cardiovascular fitness), and this group of subjects needs alternative preventative treatment to reduce the risk of developing or exacerbating cardiovascular or metabolic disease. For this non-responsive group, aggressive and earlier pharmacological intervention and/or more aggressive life style intervention, e.g. more aggressive physical therapy or dietary changes, may be the best option to help partially overcome the predisposition for low exercise training response. Currently there is no clinically proven method that has been independently validated to identify individuals who do not respond to exercise. Furthermore, pharmacological therapies aimed at enhanced aerobic fitness (e.g. PDE inhibition therapy to increase aerobic walking capacity in peripheral vascular disease patients) may be ineffective in about 20% of patients, and exposure to such drugs could be avoided if non-responders could be identified using pre-screening.

Low aerobic exercise capacity is associated with increased risks of metabolic and cardiovascular disease as well as premature death. Exercise capacity, in prospective follow-up analyses, is a stronger predictor of morbidity and mortality than other established risk factors such as hypertension or diabetes [1-5]. A notable observation in the search for relevant mechanisms which connect aerobic capacity with disease is that more humans can increase peak oxidative power through regular exercise, but some are unable to improve at all [6, 7]. Maximal aerobic capacity is commonly thought to be limited by maximal delivery of oxygen to the periphery, and hence by cardiac function [8]. Discovery of the genetic basis for this heterogeneity in responsiveness [9, 10] will provide an opportunity to identify subjects who will not benefit from exercise programs aimed at improving aerobic capacity.

Part of the heterogeneity in adaptation to regular exercise originates from variation in gene sequences that somehow influence the complex biological networks mediating the response to an aerobic exercise training stimulus. Identification of genomic markers for complex traits in humans has so far required enormous sample sizes and each single nucleotide polymorphism (“SNP”) identified seems to contribute only weakly, at least for chronic complex human diseases [11; see also, U.S. Pat. No. 7,482,117 which discloses SNPs associated with myocardial infarction]. For example, following genome-wide association analysis (GWA) in Type II Diabetes patients, 18 robust SNPs explain <7% of the total disease variance [12]. Gene network analysis generated from SNP data has improved the interpretation of the analysis [13]. However, a strategy where an expression based molecular classifier [14] is used to locate a discrete set of genes for subsequent identification of key genetic variants in combination with a set of genes generated by genomic scans and candidate gene studies has not been previously evaluated.

U.S. Patent Application Publication No. US 2008/0070247 discloses certain SNP markers to predict whether a person will respond to exercise by measuring several physiological parameters and correlating the changes with specific SNPs.

### DISCLOSURE OF INVENTION

We discovered predictor set of 29 genes using expression gene-chips whose pre-exercise expression was correlated with response to an exercise regime in term of cardiorespiratory fitness as assessed by maximal oxygen uptake, referred to herein as VO2max. This 29 predictor gene set was used to target several SNPs that were tested for similar predictive power, and 11 SNPs were discovered that could account for a large degree of the genetic variability in ability to respond to exercise. In the discovery of the 29 predictor genes, two independent muscle RNA expression data sets were generated using gene-chips (n=62 chips). One data set was used to identify, and the second set to blindly validate, an expression signature able to predict training induced increases in VO₂max, and thus finding an RNA expression-based signature useful as a diagnostic tool. To define a DNA-based diagnostic method, SNPs were genotyped in the HERITAGE Family Study (n=473) to establish if SNPs associated with the RNA expression-based predictor genes were significantly associated with gains in VO₂max. The sum of the expression of a 29 gene signature was shown to be correlated with ability to increase VO₂max with exercise. These 29 genes were subsequently used to identify SNPs that could be used to predict gains in VO₂max in the HERITAGE population. Regression analysis on the combined ‘RNA expression’ SNPs (n=25 SNPs) and 10 SNPs from candidate genes using only the HERITAGE cohort yielded 11 SNPs could explain 23% of the variance in gains in VO₂max, a value which represents about half of the estimated genetic variance for this trait. Critically, RNA expression of the genes for 10 of the 11 SNPs was not perturbed by exercise training, strongly supporting the idea that the predictor gene expression was largely pre-set by genetic factors.

Using our three step method to find biomarkers, we produced a molecular predictor that identified subjects with a range of exercise responsiveness across diverse situations (e.g., short and long term moderate intensity aerobic training and interval-based maximal exercise training regimes). This observation verified that the failure to adapt to exercise is a generalized observation and not model specific. Gains in aerobic capacity can be forecast using either a RNA or DNA SNP signature. The biomarkers that we identified, either the RNA or SNPs, can be used to predict subjects with an impaired ability to improve significantly (i.e., where significantly is defined as being beyond the error of measurement of aerobic capacity and its normal day-to-day variation) or even maintain their aerobic capacity over time, with an average ability to respond to and exercise program, and subjects with a high capacity to respond to athletic training. The low responder subjects may benefit from an alternate therapy, including a more intensive pharmacological or dietary protocol. Considering the strong relationship between maximal exercise capacity with a number of health and performance indicators, including morbidity and mortality from all causes or cardiovascular diseases, the ability to predict whether an individual will respond to regular exercise can be used, for example, to predict risk of cardiovascular disease, to design a more effective program for diabetes prevention or cardiac rehabilitation, to select recruits for physically demanding occupations (e.g., soldiers, policemen, firemen, etc.), to assess the risk and benefits if a specific drug therapy program (e.g. PDE inhibition with Cilostazol) was implemented, and to predict ability to maintain functional capacity and personal autonomy with aging using exercise therapy.

### BRIEF DESCRIPTION OF THE DRAWINGS

FIG. 1 is a schematic illustrating the three-step method used to generate the initial RNA based predictor set, to validate the RNA predictor set, and then to determine DNA SNP-based predictors.

FIGS. 2a-2c illustrate the measured changes in certain physiological characteristics of human subjects pre- and post 6 weeks of aerobic exercise training. FIG. 2a shows that the peak oxygen uptake (L·min⁻¹) increased on average by 13.7% (P<0.0001). FIG. 2b and FIG. 2c show the submaximal respiratory exchange ratio (RER) and the submaximal exercise heart rate (beats·min⁻¹), respectively, and indicate that both decreased with exercise training (P<0.0001).

FIGS. 3a and 3b show 100 genes differentially expressed in the subjects that were grouped into high and low responders to exercise based on the change in VO₂max. After 6 weeks of aerobic exercise training, these genes were observed to be differentially expressed in muscle of persons showing a high aerobic training adaptation (black columns) when compared with low-responders (white columns). Data are presented as mean percent change±SEM. *: P<0.05; **P<0.01 for the difference between low and high responders; all remaining genes P<0.07.

FIG. 4 shows the correlation between the sum score of the pre-training RNA expression level of the 29 predictor gene set of Table 4 and the measured response to exercise training in an initial cohort of volunteers (training set, Group 1; n=24; correlation (CC)=0.71; p<0.001).

FIG. 5 shows the correlation between the sum score of the pre-training RNA expression level of the 29 predictor gene set of Table 4 and the measured response to exercise training in a second, independent cohort of volunteers (test set, Group 2; n=17; correlation (CC)=0.51; p=0.02).

FIG. 6 shows the adjusted correlation between the measured response to exercise training in an independent cohort of volunteers (test set, Group 2) and the sum score of the pre-training mRNA expression level of the 29 predictor gene set of Table 4. Included in the sum score are the pre-training RNA expression levels of two genes, SVIL and NKP2, derived from the Step 3 DNA SNP predictor generation which were also validated by RNA analysis. As shown in FIG. 6, addition of pre-training mRNA expression levels of SVIL and NRP2 improved the correlation and predictability of the mRNA expression score (correlation (CC)=0.64, p=0.009), while addition of expression level of a third gene, MIPEP, did not alter performance.

FIG. 7 illustrates the assessment scale for classifying subjects based on the RNA predictor. The plot represents the quartiles of potential RNA predictor expression, and the median improvement in aerobic exercise capacity. This plot can be used to characterize subjects as belonging to one of four categories, 1) non-responder 2) poor responder 3) good responder and 4) high responder.

FIG. 8 is a flow chart illustrating potential steps in using the mRNA expression of the 29 Predictor genes to predict the response of a human subject to exercise therapy.

FIG. 9 shows the RNA expression levels of the genes as defined by the 11 predictor SNPs identified in Step 3, including the group mean expression, in Group 1 before (white bars) and following 6 weeks aerobic exercise training (black bars). RNA expression levels of 10 genes were not statistically altered by exercise training, nor was the predictor group mean value.

FIG. 10 illustrates the results of applying the predictor SNP scores to the HERITAGE Study, assigning the scores into four categories, and showing the mean unadjusted VO₂max training response for the individuals assigned to each category by their predictor SNP score.

FIG. 11 illustrates the results of applying the predictor SNP scores to the HERITAGE Study, assigning the scores into four categories, and showing the adjusted mean VO₂max training response (adjusted for age, sex, baseline body weight and baseline VO₂max) for the individuals assigned to each category by their predictor SNP score.

### MODES FOR CARRYING OUT THE INVENTION

We have discovered a method to identify an individual who will not respond well to exercise and other patterns of response level with a novel three-step process. We have also found two sets of predictive biomarkers, one based on RNA and one on DNA sequence variants. By measuring DNA obtained from blood or a number of other tissues and/or RNA in a small sample of skeletal muscle, we were able to classify individuals in a minimum of four classes of exercise training responders, ranging from those who do not respond or respond minimally to exercise to those who can be defined as high responders. After such a molecular diagnosis, a subject who would not respond to exercise can be assigned to either more aggressive pharmacological treatment or more aggressive life-style modifications, including diet and more unique intensive physical therapy (e.g., strength training). Alternate preventive measures or therapies may be more effective particularly in those who are classified as low or non-responders to regular exercise. Further, for pharmacological therapies aimed at enhancing exercise tolerance and aerobic capacity (such as Cilostazol PDE inhibition or Statin therapy for peripheral vascular disease), unnecessary exposure to drug side effects could be reduced if those non- and low-responders were identified early. Moreover, the three step method used here to identify biomarkers can be applied to identify predictive biomarkers for the ability to respond to other interventions, e.g., response to a certain drug therapy.

The invention features methods and devices that can be used to identify individuals with a lifetime risk of cardiovascular and metabolic disease since those diseases are known to be more prevalent among individuals who have a low VO₂max capacity. The RNA biomarkers relevant for this purpose were determined by obtaining a biological muscle sample from individuals prior to exercise training and grouping them according to their measured change in aerobic capacity in response to exercise. Total RNA, including mRNA and non-coding RNA (ncRNA; such as microRNAs species) was extracted from the samples and measured with one or more DNA microarrays.

Twenty-nine (29) predictor genes (assayed by 11 different sequences on the microarray) relevant for predicting response to exercise were identified based on differential RNA levels between responders and non-responders prior to the clinical intervention. These 29 genes were based on both coding and non-coding RNAs. This approach was based on RNA expression, but would also work using microRNA or protein expression. DNA SNP biomarkers were then generated by using the validated predictor biomarkers based on RNA RNA and select new genes identified in HERITAGE through sequencing only approaches to identify genes with SNPs that might segregate for the ability to respond to exercise. The RNA derived genes were thus validated in two independent studies while the sequencing based SNPs were supported using the new RNA based expression data sets (i.e. reciprocal validation). These identified SNPs were tested for correlation with the aerobic capacity response in a third study group. In the current analysis, 11 SNPs were found that were predictive of ability to respond to exercise and 10 of the 11 SNPs were associated with genes whose expression in the tissue biopsy was stable with exercise conditioning.

The RNA and DNA biomarkers can be used individually or together for classifying individuals according to their predicted response to exercise therapy. One clinical application is to select appropriate treatment for individuals identified as having or being predisposed for cardiovascular or metabolic disease. If the individual is classified as a non-responder to exercise intervention, pharmacological treatment can be started earlier and can be combined with alternative life style interventions (diet, alternative medicine modalities, relaxation techniques, etc.). Another application is to use the technologies to identify those who are talented for athletic performance in the sense that they fall into the highest responder category when exposed to aerobic training. It could also be used to identify those who are more likely to respond well to the high intensity physical training to which the candidates to armed forces are exposed to in the early screening phase. It could be used to help an individual decide which sport to participate in as low-responders are unlikely to progress in aerobic sports e.g. long distance cycling, long distance running, soccer or rowing.

“Complement” of a nucleic acid sequence or a “complementary” nucleic acid sequence as used herein refers to an oligonucleotide which is in “antiparallel association” when it is aligned with the nucleic acid sequence such that the 5′ end of one sequence is paired with the 3′ end of the other. Nucleotides and other bases may have complements and may be present in complementary nucleic acids. Bases not commonly found in natural nucleic acids that may be included in the nucleic acids of the present invention including, for example, inosine and 7-deazaguanine. “Complementarity” may not be perfect; stable duplexes of complementary nucleic acids may contain mismatched base pairs or unmatched bases. Those skilled in the art can determine duplex stability empirically or by considering factors, such as the length of the oligonucleotide, percent concentration of cytosine and guanine bases in the oligonucleotide, ionic strength, and incidence of mismatched base pairs.

When complementary nucleic acid sequences form a stable duplex, they are said to be “hybridized” and when they “hybridize” to each other or it is said that “hybridization” has occurred. Nucleic acids are referred to as being “complementary” if they contain nucleotides or nucleotide homologues that can form hydrogen bonds according to Watson-Crick base-pairing rules (e.g., G with C, A with T or A with U) or other hydrogen bonding motifs such as for example diaminopurine with T, 5-methyl C with G, 2-thiothymidine with A, inosine with C, pseudoisocytosine with G, etc. Anti-sense RNA may be complementary to other oligonucleotides, e.g., mRNA.

“Biomarker” as used herein indicates a sequence whose pre-intervention expression indicates sensitivity or resistance to a defined intervention, e.g., in this case exercise training or exercise therapy.

“DNA marker” as used herein means a variant within the DNA sequence of a gene or genomic region, i.e., a SNP, that can be correlated with an ability to respond to an intervention.

“Microarray”, including small nanoarray, as used herein means a device employed by any method that quantifies one or more subject oligonucleotides, e.g., DNA or RNA, or analogues thereof, at a time. One exemplary class of microarrays consists of DNA probes attached to a glass or quartz surface. For example, many microarrays, e.g., as made by Affymetrix, use several probes for determining the expression of a single gene. The DNA microarray may contain oligonucleotide probes that may be full-length cDNAs complementary to an RNA or cDNA fragments that hybridize to part of a RNA. The DNA microarray may also contain modified versions of DNA or RNA, such as locked nucleic acids or LNA. Exemplary RNAs include mRNA, miRNA, and miRNA precursors. Exemplary microarrays also include a “nucleic acid microarray” having a substrate-bound plurality of nucleic acids, hybridization to each of the plurality of bound nucleic acids being separately detectable. The substrate may be solid or porous, planar or non-planar, unitary or distributed. Exemplary nucleic acid microarrays include all of the devices so called in Schena (ed.), DNA Microarrays: A Practical Approach (Practical Approach Series), Oxford University Press (1999); Nature Genet. 21(1)(suppl.):1-60 (1999); Schena (ed.), Microarray Biochip: Tools and Technology, Eaton Publishing Company/BioTechniques Books Division (2000). Additionally, exemplary nucleic acid microarrays include substrate-bound plurality of nucleic acids in which the plurality of nucleic acids are disposed on a plurality of beads, rather than on a unitary planar substrate, as is described, inter alia, in Brenner et al., Proc. Natl. Acad. Sci. USA 97(4):1665-1670 (2000). Examples of nucleic acid microarrays may be found in U.S. Pat. Nos. 6,391,623, 6,383,754, 6,383,749, 6,380,377, 6,379,897, 6,376,191, 6,372,431, 6,351,712 6,344,316, 6,316,193, 6,312,906, 6,309,828, 6,309,824, 6,306,643, 6,300,063, 6,287,850, 6,284,497, 6,284,465, 6,280,954, 6,262,216, 6,251,601, 6,245,518, 6,263,287, 6,251,601, 6,238,866, 6,228,575, 6,214,587, 6,203,989, 6,171,797, 6,103,474, 6,083,726, 6,054,274, 6,040,138, 6,083,726, 6,004,755, 6,001,309, 5,958,342, 5,952,180, 5,936,731, 5,843,655, 5,814,454, 5,837,196, 5,436,327, 5,412,087, 5,405,783, the disclosures of which are incorporated herein by reference in their entireties.

Exemplary microarrays may also include “peptide microarrays” or “protein microarrays” having a substrate-bound plurality of polypeptides, the binding of an oligonucleotide, a peptide, or a protein to each of the plurality of bound polypeptides being separately detectable. Alternatively, the peptide microarray, may have a plurality of binders, including but not limited to monoclonal antibodies, polyclonal antibodies, phage display binders, yeast 2 hybrid binders, aptamers, which can specifically detect the binding of specific oligonucleotides, peptides, or proteins. Examples of peptide arrays may be found in WO 02/31463, WO 02/25288, WO 01/94946, WO 01/88162, WO 01/68671, WO 01/57259, WO 00/61806, WO 00/54046, WO 00/47774, WO 99/40434, WO 99/39210, WO 97/42507 and U.S. Pat. Nos. 6,268,210, 5,766,960, 5,143,854, the disclosures of which are incorporated herein by reference in their entireties.

“Gene expression” as used herein means the amount of a gene product in a cell, tissue, fluid, organism, or subject, e.g., amounts of DNA, RNA, or protein, amounts of modifications of DNA, RNA, or protein, such as splicing, phosphorylation, acetylation, or methylation, or amounts of activity of DNA, RNA, or proteins associated with a given gene.

The invention features methods for identifying biomarkers predictive of the response level to exercise intervention. The kits of the invention include microarrays or nanoarrays having oligonucleotide probes that are biomarkers predictive of the ability to respond to exercise that hybridize to nucleic acids derived from a muscle biopsy sample obtained from a subject. The invention also features methods of using the microarrays to determine whether a subject is a non-responder to exercise, and thus at risk of developing cardiovascular and/or metabolic disease. Thus, the methods, devices, and kits of the first part of the invention can be used to identify individuals who are likely to respond poorly, normally or highly to aerobic training. The method according to the present invention can be implemented using software that is commercially available to measure gene expression in connection with a microarray. The microarray (e.g. a DNA microarray) can be included in a kit that contains the reagents for processing a tissue sample from a subject, the microarray, the apparatus for reading the microarray, and software capable of analyzing the microarray results and predicting the response level of the subject.

The microarrays of the invention include one or more oligonucleotide probes that have nucleotide sequences or nucleotide analogues that are identical to or complementary to, e.g., at least 5, 8, 12, 20, 30, 40, 60, 80, 100, 150, or 200 consecutive nucleotides (or nucleotide analogues) of the biomarker genes or the probes listed below. The oligonucleotide probes may be, e.g., at least 5, 8, 12, 20, 30, 40, 60, 80, 100, 150, or 200 consecutive nucleotides long. The oligonucleotide probes may be deoxyribonucleic acids (DNA) or ribonucleic acids (RNA) or analogues thereof, such as LNA.

This invention may be used to predict patients who are at risk of developing cardiovascular disease and who will not respond to exercise, by using a kit that includes materials for RNA extraction from tissue samples (e.g., a sample from muscle using a tissue microsampler and an RNA stabilizing solution such as RNAlater from Ambion Inc., and an RNA extracting kit such as Trizol from Invitrogen), a kit for RNA amplification (e.g., MessageAmp from Ambion Inc), a microarray for measuring gene expression (e.g., HG-U133+2 GeneChip from Affymetrix Inc), a microarray hybridization station and scanner (e.g., GeneChip System 3000Dx from Affymetrix Inc), and software for analyzing the expression of markers as described herein (e.g., implemented in R from R-Project or S-Plus from Insightful Corp.).

For RNA analysis, cell/tissue samples are snap frozen in liquid nitrogen until processing or stabilized in RNA later at room temperature. RNA is extracted using e.g. Trizol Reagent from Invitrogen following manufacturers' instructions. RNA is amplified using e.g. MessageAmp kit from Ambion Inc. following manufacturers' instructions. microRNA is labeled using e.g. mirVana from Ambion Inc. Amplified RNA is quantified using a human microarray chip, e.g. HG-U133+2 GeneChip from Affymetrix, Inc., and compatible apparatus to read the resulting array, e.g. GCS3000Dx from Affymetrix. MicroRNA can be quantified using Affymetrix chips containing probes for microRNAs. The resulting gene expression measurements are further processed by methods otherwise known in the art, e.g., as described below in Example 1.

For prediction to exercise response less than 30 biomarkers were shown sufficient to give an accurate prediction. Given the relatively small number of biomarkers required, other procedures, such as quantitative reverse transcriptase polymerase chain reaction (qRT-PCR), may be performed to measure with greater precision the level of biomarkers expressed in a sample. This will provide an alternative to or a complement to DNA microarrays. qRT-PCR may be performed alone or in combination with a microarray as described herein. Procedures for performing qRT-PCR are well known and described in several publications, e.g., U.S. Pat. No. 7,101,663 and U.S. Patent Application Nos. 2006/0177837 and 2006/0088856.

In addition, we have identified a set of 11 SNPs that are predictive of response to aerobic exercise training. A SNP may be screened from DNA extracted from blood or any other biological sample obtained from an individual. One embodiment of the present invention involves obtaining nucleic acid, e.g. DNA, from a blood sample of a subject, and assaying the DNA to determine the individuals' genotype of a combination of the marker genes associated with response to exercise. Other less intrusive samples could be taken, e.g., use of buccal swabs, saliva, or hair root. Genotyping preferably is performed using a gene array methodology, which can be readily and reliably employed in the screening and evaluation methods according to this invention. A number of gene arrays are commercially available for use by the practitioner, including, but not limited to, static (e.g. photolithographically set), suspended (e.g. soluble arrays), and self assembling (e.g. matrix ordered and deconvoluted). The SNPs that are biomarkers for the response to exercise form the basis for a kit comprising SNP detection reagents, and methods for detecting the SNPs by employing detection reagents. An array can easily be made that encompasses the 11 SNPs. Many such detection reagents or assays are known, including those discussed in U.S. Pat. No. 7,482,117.

The present invention provides a screening method to allow the identification of subsets of individuals who have specific genotypes and who are more or less likely to respond favorably to exercise. For example, a screening method involves obtaining a sample from an individual undergoing testing, such as a blood sample, and employing an assay method, e.g. the array system for the marker gene variants as described, to evaluate whether the individual has a genotype associated with a low or a high response to exercise. Then using methods identified below, the person may be assigned to a category of response level to exercise. This screening method can also be used to identify individuals with a higher risk of either cardiovascular or metabolic disease, and to identify individuals gifted for athletic performance or high performing recruits for occupations requiring high aerobic capacity.

### Example 1

### Materials and Methods

### Study Groups

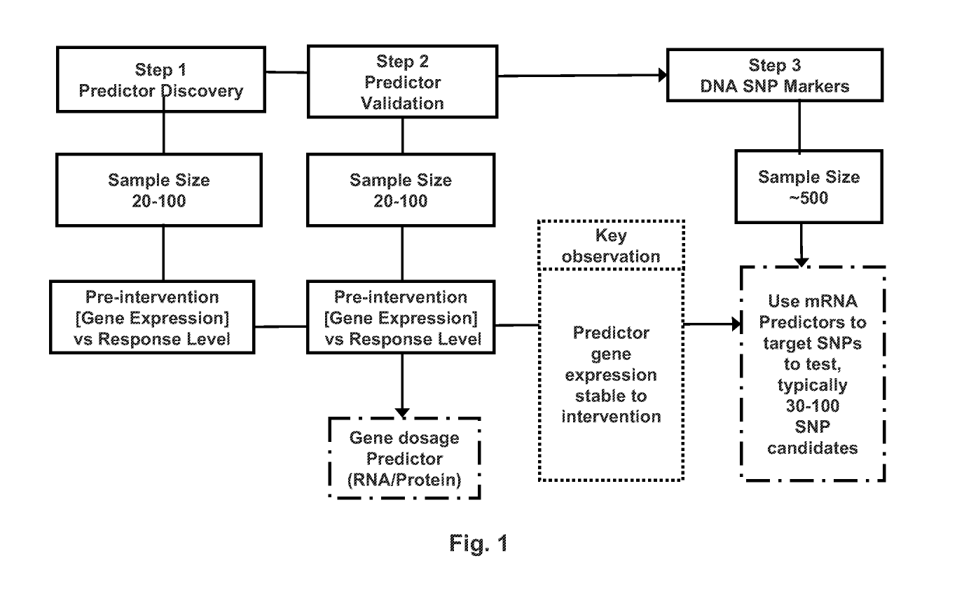

Three independent clinical studies were used. The first (Group 1) was used to generate the predictor set of biomarkers, the second (Group 2) to independently validate the predictor set of biomarkers, and the third (Group 3) to assay for links between the predictor biomarkers and other candidate genes and genetic variation as seen in DNA SNPs, the DNA markers (FIG. 1). Each clinical study is based on supervised endurance training program with primarily sedentary or recreationally active subjects of differing levels of physical fitness which establishes that the results can be applied broadly to various types of aerobic exercise therapy and subjects.

Group 1 for producing molecular predictor. Twenty-four healthy sedentary Caucasian males took part in the study. Their mean (with the range) age, height and weight are given in Table 1. Body mass did not change during the study period (78.6±2.7 kg vs 78.8±2.6 kg). Resting blood pressure (systolic/diastolic (mm Hg)) and heart rate (beats·min⁻¹) were 126/72 and 66±3, respectively. The study was approved by the ethics committee of the Karolinska Institute, Stockholm, Sweden, and informed consent was obtained from each of the volunteers. Subjects abstained from strenuous exercise during the three weeks prior to obtaining pre-training muscle biopsies (vastus lateralis). Subjects trained under supervision on a cycle ergometer four times a week (45 min) at 75% of their pre-training maximal aerobic capacity (peak VO₂) for six weeks. Post-training biopsies were taken 24 h following the last training session. Physiological measurements and muscle biopsies were performed as previously described [15, 16]. All physiological parameters were derived from a minimum of two assessments on separate days. Peak VO₂ was determined using a cycle ergometer (Rodby, Sweden). An incremental protocol was combined with continuous analysis of respiratory gases (Sensormedic). At exhaustion, the respiratory exchange ratio and heart rate exceeded 1.10 and 190 beats·min⁻¹, respectively. Total amount of work done in 15 min of cycling was determined using a self-paced protocol (Lode, Netherlands, test-re-test variability<5%). Submaximal physiological parameters were determined during two separate 15 min constant load submaximal cycling sessions (both at 75% of pre-training peak VO₂). Following six weeks training, two groups were identified from the original 24 subjects: a high responder group (n=8; the top ⅓ responders) and a low responder group (n=8; the bottom ⅓ responders). Subjects were assigned to groups after being ranked based on the % change in maximal aerobic power. This ranking process occurred prior to any biochemical or molecular analysis. The response to exercise training in the high and low responders was similar to results a much larger scale study (n=1000), the HERITAGE study [17].

TABLE 1
Group 1 Subject Characteristics

| | Pre-training |
|---|---|
| | (mean ± sem) |
| Body Mass (kg) | 78.6 ± 2.7 |
| Age (y) | 23 ± 1 |
| Height (m) | 1.82 ± 0.02 |
| VO₂max (L · min⁻¹) | 3.71 ± 0.55 |

Values are mean (SE)

Group 2 for validating molecular predictor. Seventeen young active Caucasian subjects (Table 2) trained on a cycle ergometer (Monark 839E, Monark Ltd, Varberg, Sweden) 5 times a week for 12 weeks. The training load was incrementally increased during the study such that these active/trained subjects trained at a higher intensity and volume than Group 1 subjects. As part of the training, the subjects performed a peak power (Pₘₐₓ) test every Monday in order to determine the intensity of the training for the following days. The Pₘₐₓ-test was performed the same way as the VO₂max-test without measuring oxygen consumption. On Tuesdays, the training consisted of 10, 3-min intervals at 85% Pₘₐₓ with 3-min intervals at 40% Pₘₐₓ in between. The next day the training consisted of 60 min at 60% Pₘₐₓ. On Thursdays, subjects performed 5, 8-min intervals at 75% Pₘₐₓ with a 4-min interval at 40% Pₘₐₓ in between. On Fridays, subjects cycled for 120 min at 55% Pₘₐₓ continuously. The first six weeks, the duration of each training session was increased by 5% every week. During the last six weeks, the duration remained the same but the relative intensity was increased 1% per week. The compliance to training was ˜100%.

TABLE 2
Group 2 Subject Characteristics

| | Pre-training |
|---|---|
| | (mean ± SD) |
| Age (y) | 29 ± 6 |
| Body Mass (kg) | 81.8 ± 9.0 |
| Height (m) | 1.8 ± 0.5 |
| VO₂max (L · min⁻¹) | 4.1 ± 0.5 |

Values are mean (SE)

Group 3 to find DNA SNP Biomarkers: HERITAGE Family Study aerobic training program. The study cohort was from the HERITAGE Family Study and consisted of 473 Caucasian subjects (230 males and 243 females) from 99 nuclear families who completed at least 58 of the prescribed 60 exercise training sessions. The study design and inclusion criteria have been described previously [18]. To be eligible, the individuals were required to be in good health, i.e., free of diabetes, cardiovascular diseases, or other chronic diseases that would prevent their participation in an exercise training program. Subjects were also required to be sedentary, which was defined as not having engaged in regular physical activity over the previous 6 months. Individuals with a resting systolic blood pressure (SBP) greater than 159 mmHg or a diastolic blood pressure (DBP) more than 99 mmHg or taking medication for hypertension, dyslipoproteinemia or hyperglycemia were excluded. Other exclusion criteria are described in a previous publication [18]. The baseline characteristics are given in Table 3. The prevalence of overweight and obesity was 30.8% and 19.3%, respectively. The study protocol had been approved by each of the Institutional Review Boards of the HERITAGE Family Study research consortium. Written informed consent was obtained from each participant.

TABLE 3
Baseline characteristics of the HERITAGE Family Study subjects.

| | All | Men | Women |
|---|---|---|---|
| N | 473 | 230 | 243 |
| Age, years | 35.7 (14.5) | 36.7 (15.0) | 34.8 (14.0) |
| BMI, kg/m² | 25.8 (4.9) | 26.6 (4.9) | 24.9 (4.8) |
| VO2max, L/min | 2.46 (0.7) | 2.03 (0.6) | 1.91 (0.4) |
| VO2max, ml/kg/min | 33.2 (8.8) | 37.0 (9.0) | 29.5 (6.9) |

Values are mean (SD)

The exercise intensity of the 20-week program was customized for each participant based on the heart rate (HR)—VO₂ relationship measured at baseline [19]. During the first two weeks, the subjects exercised at a HR corresponding to 55% of the baseline VO₂max for 30 minutes per session. Duration and intensity of the sessions were gradually increased to 50 minutes and 75% of the HR associated with baseline VO₂max, which were then sustained for the last six weeks. Frequency of sessions was three times per week, and all exercise was performed on cycle ergometers in the laboratory. Heart rate was monitored during all training sessions by a computerized cycle ergometer system (Universal FitNet System), which adjusted ergometer resistance to maintain the target HR. Trained exercise specialists supervised all exercise sessions. Before and after the 20-week training program, each subject completed three cycle ergometer (SensorMedics Ergo-Metrics 800S, Yorba Linda, Calif.) exercise tests on separate days: a maximal exercise test (Max), a submaximal exercise test (Submax) and a submaximal/maximal exercise test (Submax/Max). The Max test started at 50 W for 3 min, and the power output was increased by 25 W every 2 min thereafter to the point of exhaustion. For older, smaller, or less fit subjects, the test was started at 40 W and increased by 10 to 20 W increments. Based on the results of the Max test, the Submax test was performed at 50 W and at 60% of the initial VO₂max. Finally, the Submax/Max test was started with the Submax protocol and progressed to a maximal level of exertion. For all tests, VO₂, VCO₂, expiratory minute ventilation (VE) and tidal volume (TV) were determined every 20 s and reported as a rolling average of the three most recent 20-s values. All respiratory phenotypes were measured using a SensorMedics 2900 metabolic measurement cart. VO₂max was defined as the mean of the highest VO₂ values determined on each of the maximal tests, or the higher of the two values if they differed by more than 5%.

### Example 2

### Materials and Methods

### RNA and DNA Analyses

Affymetrix Microarray process. Total RNA was extracted from frozen muscle samples taken from Groups 1 and 2. Two samples were available for each subject, one taken pre-exercise and a second one taken post-exercise. RNA was extracted using TRIzol reagent. Frozen pieces were homogenized for 60 s in 1 ml of TRIzol using a 7 mm Polytron aggregate (PT-DA 2107, Kinematica AG, Switzerland) adapted to a Polytron homogenizer (PT-2100) running at maximum speed. RNA concentration and quality were controlled using a Bioanalyser. In-vitro transcription (IVT) was conducted using the Bioarray high yield RNA transcript labeling kit (P/N 900182, Affymetrix, Inc.). Unincorporated nucleotides from the IVT reaction were removed using the RNeasy column (QIAGEN Inc, U.S.A.). Group 2 in vitro transcription was performed using MessageAmp II Biotin Enhanced aRNA kit (Ambion, Inc). The effect of the IVT kit was assessed by processing two samples with the Affymetrix kit used for Group 1. Hybridization, washing, staining and scanning of the arrays were performed according to manufacturer's instructions (e.g., Affymetrix, Inc. http://www.affymetrix.com/). As a means to control the quality of the individual arrays, all were examined using hierarchical clustering and NUSE to identify outliers prior to statistical analysis in addition to standard quality assessments including scaling factors and housekeeper 5′/3′ ratios.

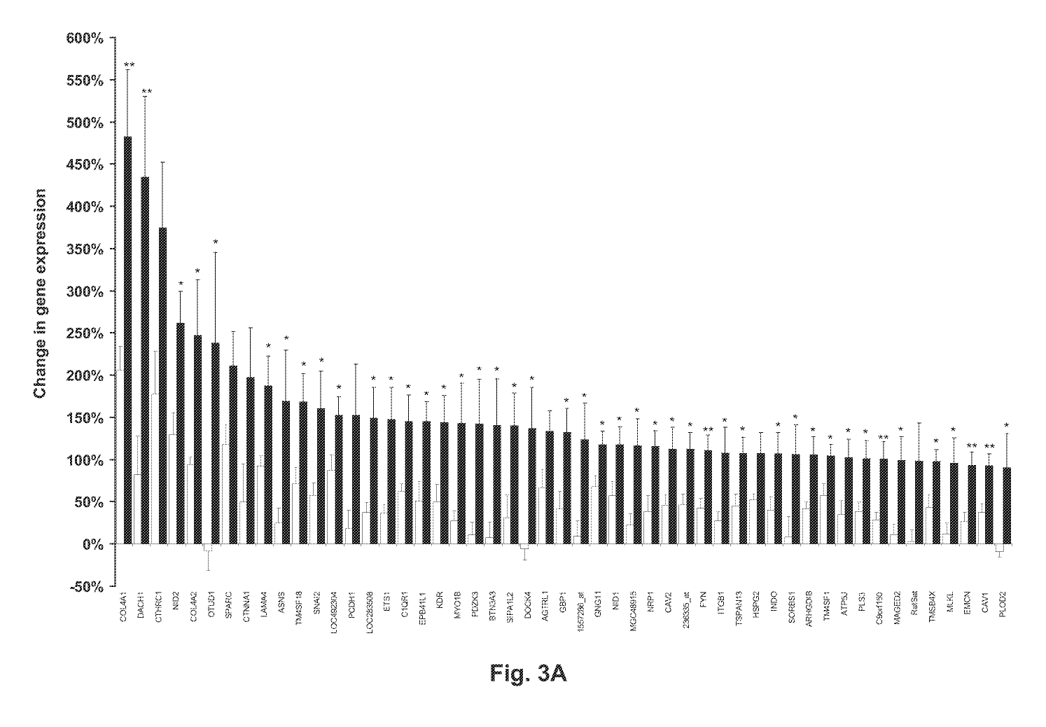

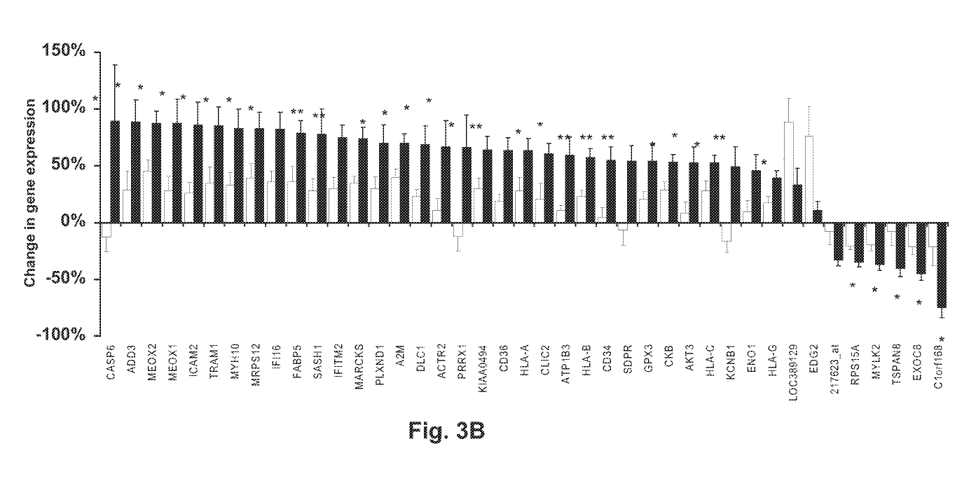

General array analysis methods. The microarray data was subjected to global normalization using MAS5.0, and present-absent calls were used to improve the sensitivity of the differential gene expression analysis by improving the power while potentially removing some genuinely expressed genes by known methods [20]. We chose to retain probe sets for which a minimum of 25% of the chips indicated a ‘present’ detection, on the basis that there will be subject-to-subject variability and that some genes may only be expressed either before or following training. The normalized log 2-file was analyzed with the Significance Analysis of Microarray (SAM) in R (http://www-stat.stanford.edu/˜tibs/SAM/) [9]. SAM provides an estimate of the false discovery rate (FDR), which represents the percentage of genes that could be identified by chance, and is comparable to a P-value corrected for the number of initial comparisons, a process called multiple testing correction. For the data presented in FIGS. 3A and 3B, genes were considered significantly changed following training, when a delta value corresponding to the number of false significant genes of 5% (q-value) and an average fold change of 1.5 were achieved. We have previously demonstrated that it can be difficult to predict the impact of applying arbitrary filtering criteria prior to statistical analysis [21]. We therefore relied on several statistical models to present, analyze, and interpret the data. We also used a web-based bioinformatics tool, Ingenuity pathway analysis (IPA, http://www.ingenuity.com).

Production of a Quantitative predictor of response to training: A quantitative predictor of response to training was developed by correlating measured change in VO₂max after training to expression levels of RNA from a muscle biopsy obtained prior to training. Data from the Affymetrix microarray chip were gathered according to manufacturer's direction into “CEL” files and then were logit normalized, and an expression index calculated using the li-wong method [22]. The normalisation settings for the training set files were re-used for the validation data set to increase comparability. To calculate a correlation between VO₂max response and expression level for a given gene or probeset, the Pearson correlation for each affymetrix perfect match probe in the probeset was used and retained to generate the median correlation for that gene or probeset. If the median correlation exceeded 0.3, the entire probeset was retained as correlated. Correlated probesets were identified 24 times on the 24 sample training set, each time leaving one sample out of the calculation. Probesets were ranked according to how many out of 24 times they were selected as having a median correlation above 0.3. The procedures described above were implemented using R software freely available from R-Project and supplemented with packages available from Bioconductor, or other known statistical programs.

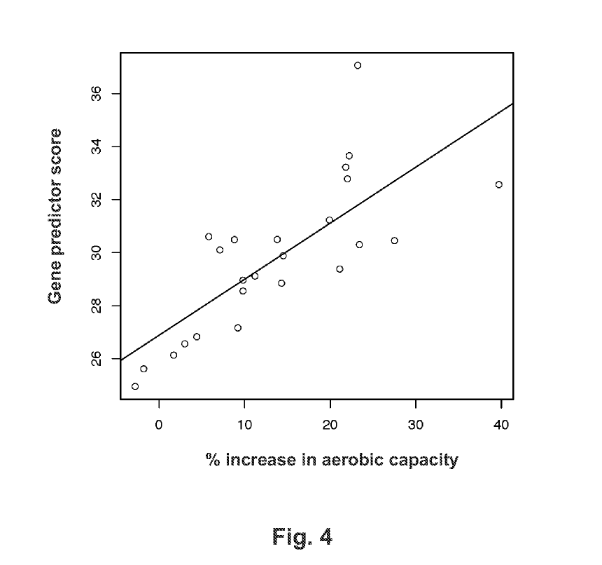

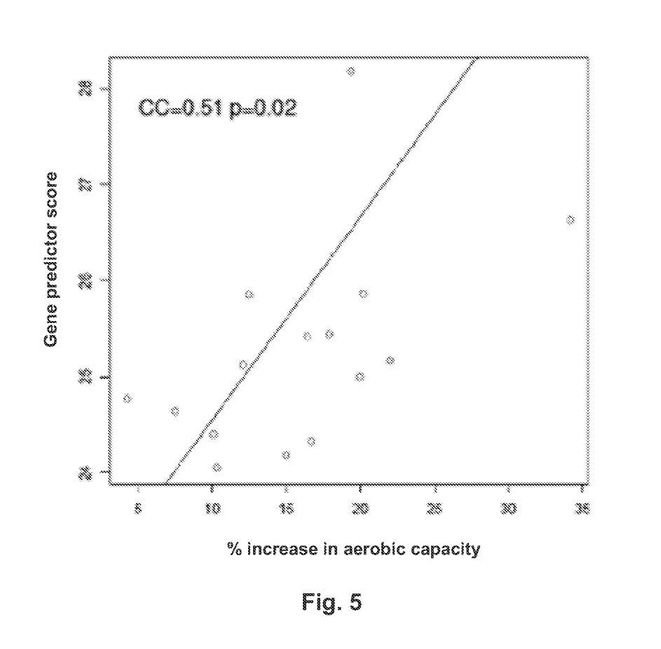

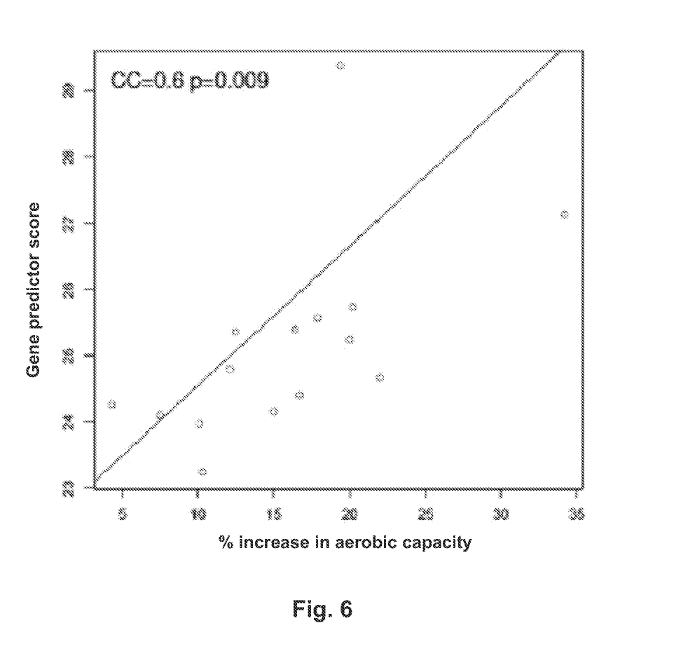

The top 29 genes that were selected 22 or more times out of 24 runs were those which gave the best correlation to VO₂max on the training set (Group 1) and are shown below in Table 4. For each individual a gene predictor score was calculated using the sum of the normalized expression values using the li-wong expression method. The logit normalized model based expression index [24] values for each of the 29 genes were then centered and scaled over the 24 subjects in Group 1 (so each subject's expression values could be directly compared), and correlation plots were generated comparing this expression metric with the measured change in VO₂max (FIG. 4). The expression value of each of the 29 genes was then determined in Group 2, and the sum of the expression of the 29 genes in Group 2 was correlated to the measured change in VO₂max as before by an observer blinded to sample identity. These results are shown in FIG. 5. To allow comparison between cohorts that had a different baseline VO₂max, the percent change in VO₂max was used. Finally, for genes and SNPs identified in the Group 3 study (see below), the genetic association data was validated using expression-based correlation analysis in the Group 2 blind validation data set. Two of the validated SNP genes were then added to the 29 gene predictor to test performance in the validation data set of Group 2 (FIG. 6).

Genotype validation and extension of the expression based predictor. Linkage disequilibrium (LD) cluster tagging single nucleotide polymorphisms (tagSNPs) were selected from the Caucasian data set of the International HapMap consortium (date of release 23 Mar. 2008). Target areas for the SNP selection for the 29 predictor genes were defined as the coding region of each gene plus 20 kb upstream of the 5′ end and 10 kb downstream of the 3′ end of the coding region. TagSNPs were selected using the pairwise algorithm of the Tagger program [24]. Minor allele frequency was required to be greater than 10%, and the pairwise linkage disequilibrium threshold for the LD clusters was set to r²≧0.80.

Genomic DNA was prepared from permanent lymphoblastoid cells from blood collected from the Group 3 subjects with a commercial DNA extraction kit (Gentra Systems, Inc., Minneapolis, Minn.). The tagSNPs were genotyped using a customized array made by Illumina (San Diego, Calif.) based on the SNPs selected above, using GoldenGate chemistry and Sentrix Array Matrix technology on the BeadStation 500GX. Genotype calling was done with Illumina BeadStudio software, and each call was confirmed manually. For quality control purposes, each 96-sample array matrix included one sample in duplicate and 47 samples were genotyped in duplicate on different arrays. In addition, six CEPH (Centre d'Etude du Polymorphisme Humain) control DNA samples (NA10851, NA10854, NA10857, NA10859, NA10860, NA10861 and all samples included in the HapMap Caucasian panel) were genotyped. Concordance between the replicates as well as with the SNP genotypes from the HapMap database was 100%.

A chi-square test was used to verify whether the observed genotype frequencies at the loci of the SNPs were in Hardy-Weinberg equilibrium. Associations between the individual tagSNPs and cardiorespiratory fitness phenotypes were analyzed using a variance components and likelihood ratio test based procedure in the QTDT software package [25]. The total association model of the QTDT software utilizes a variance-components framework to combine a phenotypic means model and the estimates of additive genetic, residual genetic, and residual environmental variances from a variance-covariance matrix into a single likelihood model. The evidence of association is evaluated by maximizing the likelihoods under two conditions: the null hypothesis (L₀) restricts the additive genetic effect of the marker locus to zero (βₐ=0), whereas the alternative hypothesis does not impose any restrictions on βₐ. The quantity of twice the difference of the log likelihoods between the alternative and the null hypotheses (2[ln(L₁)−ln(L₀)]) is distributed as χ² with 1 df (difference in number of parameters estimated). VO₂max training responses were reported as unadjusted scores and as values adjusted for age, sex, baseline body weight and baseline value of VO₂max. Differences in allele and genotype frequencies between top and bottom quartiles of VO₂max training response distribution (defined using sex and generation-specific quartile cut-offs) were tested using the case-control procedure (Proc Casecontrol) of the SAS version 9.1 Statistical Software package. Finally, the total contribution of the SNPs on VO₂max training response was tested using multivariate regression analysis. Backward elimination was used to filter out redundant SNPs due to strong pair-wise LD. Then, the SNPs retained by the backward elimination model were analyzed using a stepwise regression model.

### Example 3

### Three Step Model Used to Find Biomarkers that Predict Responsiveness to Intervention Therapy

FIG. 1 illustrates the analysis strategy and approximate sample sizes required to generated a molecular predictor based on pre-treatment gene expression, followed by validation, and then by identification of genetic variation. Similar sample sizes can be used to both generate the initial gene predictor set and to independently validate the observation. Gene expression can be measured using RNA, miRNA, or proteins, or other known methods. In the current work, RNA was measured and the sample sizes were 24 and 17 for the initial group and the validation group, respectively. The initial expression classifier, be it RNA or protein, can, for example, be derived from tissue or blood. The candidate genes can thereafter (Step 3) be used to locate genetic variants that are also correlated with the measured physiological function. This final step was based on a sample size of 473. These sample sizes are markedly lower than have been reported for significant p-values during a genome-wide search for SNPs due to much reduced multiple testing. The sample sizes are sufficiently low to be cost-effective, and thus useful for finding biomarkers for other physiological responses, for example, for pharmaceutical drug response screening. In addition, the method identified SNPs located in genes whose expression was largely independent of exercise conditioning. This predictor set is thus applicable across a wide range of subjects.

### Example 4

### Physiological Adaptation to Aerobic Exercise Training is Highly Variable in Humans

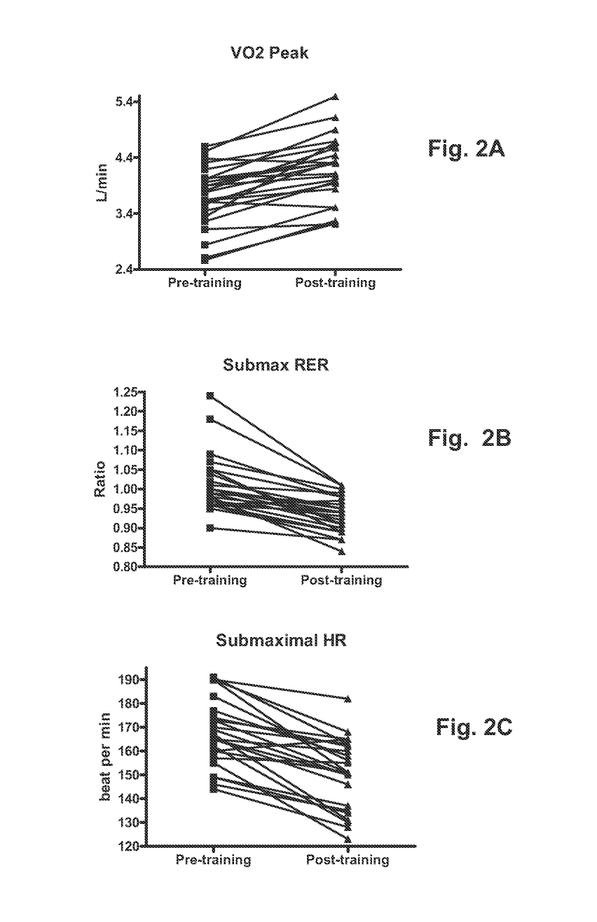

In the Group 1 subjects, the average peak oxygen uptake (aerobic capacity; peak VO₂) improved 13.7±2.1% (P<0.0001) after 6 weeks of supervised training (FIG. 2a). The individual changes varied from a 27.5% improvement to a −2.8% decline consistent with the initial hypothesis that some otherwise healthy subjects do not improve aerobic fitness with training. During submaximal cycling (at 75% of pre-exercise peak VO₂), respiratory exchange ratio (RER) was 1.01±0.07 prior to training and 0.91±0.05 after training (P<0.0001) indicating a shift towards lipid oxidation, while submaximal heart rate was 10±1% (P<0.0001) lower after 6 weeks of training (FIGS. 2b and 2c).

### Example 5

### Identification of a Human Exercise mRNA Transcriptome

An Affymetrix U133+2 chip was used to generate data for all subjects in Group 1 (n=24, 48 chips), and normalized using MAS5.0. A ‘present call’ filter of 12 present from 48 chips was applied yielding 20,194 probe sets. Only those subjects that demonstrated an increase in aerobic capacity were entered into the initial global analysis (40 chips from a possible 48). We found >900 up-regulated probe sets (false-discovery-rate (FDR)<4.5%) with a 1.5 fold change (FC) or greater with MAS5.0 normalized data. Very few probe sets were down-regulated in human skeletal muscle following aerobic training. A conservative list of 100 genes (from the ˜1000 modulated genes) was identified (named the Training Responsive Transcriptome or “TRT”), which were modulated to a greater extent in those subjects who demonstrated the greatest increase in aerobic capacity (n=8), compared with those showing the least aerobic capacity gain (n=8). These 100 genes and the changes in gene expression are shown in FIG. 3a and FIG. 3b. This clearly indicates that high and low responders have a different molecular response.

### Example 6

### Quantitative Predictor of Response to Training

A quantitative predictor set of 29 genes of response to training was developed by correlating measured change in peak VO₂max after training to expression levels in a muscle biopsy obtained prior to training in the Group 1 subjects. The expression level for each gene is based on the results from a specific probe-set used on the Affymetrix genechip array. Each probe set is composed of 11 oligonucleotide probes, and each probe sequence is the antisense sequence to the biological RNA that is detected. Genes with a positive correlation of 0.3 or more to the measured change in VO2max in the training set of 24 subjects were identified. This correlation analysis was repeated 24 times in the training set of 24 subjects, each time leaving a different subject out. Genes were ranked according to the number of times they were found correlated (up to 24 times). The 29 genes (Table 4) that were found to correlate 22 times or more performed best in predicting VO2max in the training set when their expression values were summed. This correlation is shown in FIG. 4 (CC=0.71, p<0.001). For these 29 genes, the Affymetrix “probeset identifier” is provided in Table 4 along with the probe-set sequences. In addition, the full sequence for each gene is readily available from public databases, e.g., NCBI Entrez Gene data base (http://www.ncbi.nlm.nih.gov/gene). To find that sequence one would take the probe-set sequence and produce the complimentary matching sequence and BLAST (a search tool) this sequence at NCBI. Alternatively, one can take the unique probe-set sequence and search at http://www.affymetrix.com/index.affx. This site will provide an automatic link to the NCBI.

TABLE 4
List of Probes, Corresponding Gene Names,
Gene Sequences and SEQ ID NOs.

| | | Detection probe-set | SEQ |
|---|---|---|---|
| | Affymetrix | sequence (Antisense to | ID |
| Gene name | Probe name | the biological target) | NO. |
| SLC22A3 | 1570482_at | TTAGCACCACAAGAATACACAACAC | 37 |
| | | AGAGATATTCAACATTCATGGATAG | 38 |
| | | GATGTCAGTTCTTCCCAACTTGATG | 39 |
| | | GTTCTTCCCAACTTGATGTATATAT | 40 |
| | | AAATCCTACAGAGTTATTTTGTGGA | 41 |
| | | GAATAGCCAACGCAGTACTGAAGGA | 42 |
| | | CCAGAGGACTGGCACTACTTAACGT | 43 |
| | | TGGCACTACTTAACGTCAAGACTTA | 44 |
| | | TCAAGACTTACCGTAAAGCGACAGT | 45 |
| | | GTAAAGCGACAGTAATCACGACAGT | 46 |
| | | ATAGACCTCTACCAATAGTTCAGTG | 47 |
| DNAJB1 | 200666_s_at | CCCTTGATGGTCTGGGAGCCTGGCC | 48 |
| | | ATGTCCTCACTTTGTGGGTCACACT | 49 |
| | | GGTCACACTCTTTACATTTCTGTAA | 50 |
| | | GTAAGGCAATCTTGGCACACGTGGG | 51 |
| | | GCACACGTGGGGCTTACCAGTGGCC | 52 |
| | | TCCTTTTGAATTTTGCACAGCCCTA | 53 |
| | | CAGCCCTAGATACAATCCCTTTTGA | 54 |
| | | GGAGCACTGTGGAACGTCTGTAAAT | 55 |
| | | TTGGTGTACACTCAAAACCTGTCCC | 56 |
| | | GCAGCCAGTGCTCTCTGTATAGGGC | 57 |
| | | TCCAGTGCTCAGACCTTTAGACTCA | 58 |
| IER2 | 202081_at | GCGTTTCCAACCTCGGAGAATTCCA | 59 |
| | | GTATAAGCGGTCATCGTTGCGTCAT | 60 |
| | | GGGTGTGGGCCTGGAGGAAGGTCCT | 61 |
| | | GAGAGTGGCCTGAGTTACTTCACCC | 62 |
| | | CGCGTGCTGCTGGTTAATGTCCCGC | 63 |
| | | GGACTGATCTACTTTCACATTCTCA | 64 |
| | | GCATTAGAGGTCCCCAGTAGGTTCC | 65 |
| | | CAGCCGAGAAGTTCCTGGTCTGAAT | 66 |
| | | GTTTCTGAGGGTCTGCTTTGTTTAC | 67 |
| | | GTTTACCTTTCGTGCGGTGGATTCT | 68 |
| | | TCCGTCTACCTGGCGTTTTGTTAGA | 69 |
| AMOTL2 | 203002_at | GGGGTGAAACACCCACATGGCAGCC | 70 |
| | | CACATGGCAGCCTGCTAGCAGCAGT | 71 |
| | | CTGGTCTTAAAGAGTCCCTCACTTC | 72 |
| | | TCAGCCCCAGGAGCTATTGGTGGGT | 73 |
| | | TTTTTAGTTCTCCTTGATTCTTTGT | 74 |
| | | TATCGTTTTTAGGTTTGGTATGTGT | 75 |
| | | ATTTCCATGGTTCCTCAAGTTTCCT | 76 |
| | | ATACATTTGGTTCATGTGCATTGTT | 77 |
| | | TTTTTGTGCTGTGAACATTTTCTGC | 78 |
| | | GTGTCTGTATGTTTAAGTTATCGTA | 79 |
| | | ATGGCTGTTTTGTTATGCCACCCTG | 80 |
| IL32 | 203828_s_at | ACCTGGAGACAGTGGCGGCTTATTA | 81 |
| | | GGCTTATTATGAGGAGCAGCACCCA | 82 |
| | | AAGAGATGGATTACGGTGCCGAGGC | 83 |
| | | TACGGTGCCGAGGCAACAGATCCCC | 84 |
| | | ATCCCCTGTCCCGGATGTTGAGGAT | 85 |
| | | TCCCGGATGTTGAGGATCCCGCAAC | 86 |
| | | CCCGCAACCGAGGAGCCTGGGGAGA | 87 |
| | | TGAGATGGTTCCAGGCCATGCTGCA | 88 |
| | | CTGCTCTCTGTCAGAGCTCTTCATG | 89 |
| | | CTGACACCCCAGAAGTGCTCTGAAC | 90 |
| | | ATGAAGATACTGACACCACCTTTGC | 91 |
| ENOSF1 | 204143_s_at | CCTCTGTGAACTGGTGCAGCACCTG | 92 |
| | | ACATATCAGTTTCTGCAAGCCTTGA | 93 |
| | | GTGTGTGAGTATGTTGACCACCTGC | 94 |
| | | GTATGTTGACCACCTGCATGAGCAT | 95 |
| | | GCATGAGCATTTCAAGTATCCCGTG | 96 |
| | | GTATCCCGTGATGATCCAGCGGGCT | 97 |
| | | GTAAAGAAACACCAGTATCCAGATG | 98 |
| | | TCCTTCCTGCTCAAGAAAATTAAGT | 99 |
| | | AAATCCTACCGATCAAGATGAGTTC | 100 |
| | | GTTCAGCTAGAAGTCATACCACCCT | 101 |
| | | CATACCACCCTCAGGAATCAGCTAA | 102 |
| ID3 | 207826_s_at | GAACTTGTCATCTCCAACGACAAAA | 103 |
| | | AAAAGGAGCTTTTGCCACTGACTCG | 104 |
| | | CCTCCAGAACGCAGGTGCTGGCGCC | 405 |
| | | GGAAGCCGGACGGCAGGGATGGGCC | 106 |
| | | GGTGCTCAGGAGCGAAGGACTGTGA | 107 |
| | | GTGGCCTGAAGAGCCAGAGCTAGCT | 108 |
| | | GGTCTTTTCAGAGCGTGGAGGTGTG | 109 |
| | | GAAGGAGTGGCTGCTCTCCAAACTA | 110 |
| | | CTGCTCTCCAAACTATGCCAAGGCG | 111 |
| | | ACTATGCCAAGGCGGCGGCAGAGCT | 112 |
| | | TTGGAGAAAGGTTCTGTTGCCCTGA | 113 |
| CPVL | 208146_s_at | GAAATTTTTGTCACTCCCAGAGGTG | 114 |
| | | GACAAGCCATCCACGTGGGGAATCA | 115 |
| | | ACAGTACAGTCAGTTAAGCCATGGT | 116 |
| | | TAAGGTTCTGATCTACAATGGCCAA | 117 |
| | | CAATGGCCAACTGGACATCATCGTG | 118 |
| | | ACAGAGCACTCCTTGATGGGCATGG | 119 |
| | | GTGAAGTGGCTGGTTACATCCGGCA | 120 |
| | | TTACATCCGGCAAGCGGGTGACTCC | 121 |
| | | GGGTGACTCCCATCAGGTAATTATT | 122 |
| | | GACATATTTTACCCTATGACCAGCC | 123 |
| | | TATGTTGGATAAACTACCTTCCCGA | 124 |
| METTL3 | 209265_s_at | GAAGACAAATCAACTGCAACGCATC | 1259 |
| | | AACGCATCATTCGGACAGGCCGTAC | 126 |
| | | GGCCGTACAGGTCACTGGTTGAACC | 127 |
| | | ATCCCCAAGGCTTCAACCAGGGTCT | 128 |
| | | GGTTCGTTCCACCAGTCATAAACCA | 129 |
| | | TATCTCCTGGCACTCGCAAGATTGA | 130 |
| | | GGACGACCACACAATGTGCAACCCA | 131 |
| | | AATGTGCAACCCAACTGGATCACCC | 132 |
| | | GGATCACCCTTGGAAACCAACTGGA | 133 |
| | | TGGATGGGATCCACCTACTAGACCC | 134 |
| | | GCCATGGCTCTGTAAGCTAAACCTG | 135 |
| BTAF1 | 209430_at | TGCATAGATGTACCTATCCTGCACC | 136 |
| | | GTACCTATCCTGCACCCAAAAAGGT | 137 |
| | | ATCATGTAGTTATACTGGGCAGCAA | 138 |
| | | GGGCATGAGGCTGATTACTCAATGG | 139 |
| | | TACAGGTAATAAACATCCCCAAGGT | 140 |
| | | GTGGCTGGCCATACACATAGGCATC | 141 |
| | | ATCAGTTTAACAACCATCAGACCTC | 142 |
| | | AGACCTCAGCTGTACAATAACAGGT | 143 |
| | | GTTCTGCAGCATTTAGACATTTGTC | 144 |
| | | TTAGCTTTGACAACCATACTGTAAC | 145 |
| | | GTAACATTAAACCTAGCATTCCACA | 146 |
| SCN3A | 210432_s_at | AAACCTGTGCTTGATCTGACATTTG | 147 |
| | | GCATGATTCACCAAGCAGTACTACA | 148 |
| | | GTTCACATGTTCCAACTTTCAGGTT | 149 |
| | | GTAACCACCTACAATAGCTTTCAAT | 150 |
| | | TTCAATTTCAATTAACTCCCTTGGC | 151 |
| | | AACTCCCTTGGCTATAAGCATCTAA | 152 |
| | | GCATCTAAACTCATCTTCTTTCAAT | 153 |
| | | GCTATCTCCTAATTACTTGGTGGCT | 154 |
| | | GAACCCTTGGATTTATGTGAGGTCA | 155 |
| | | GGTCAAAACCAAACTCTTATTCTCA | 156 |
| | | ATGTATTTCATAATTCTCCCATAAT | 157 |
| MAST2 | 211593_s_at | CTCCACCTCTGGGAAGCTGAGCATG | 158 |
| | | GAGCATGTGGTCCTGGAAATCCCTT | 159 |
| | | GAAATCCCTTATTGAGGGCCCAGAC | 160 |
| | | CAGACAGGGCATCCCCAAGCAGAAA | 161 |
| | | GCATCCCCAAGCAGAAAGGCAACCA | 162 |
| | | GGCAACCATGGCAGGTGGGCTAGCC | 163 |
| | | AACCTGTCTCCCAGGGAGCAGGGGA | 164 |
| | | GGCCCATCCATCTTATGAGGATCCC | 165 |
| | | GGCTGGCTATGGGAGTCTGAGTGTG | 166 |
| | | GGAGTCTGAGTGTGCACAAGCAGTG | 167 |
| | | GTGAAAGAGGATCCAGCCCTGAGCA | 168 |
| DEPDC6 | 218858_at | GAACTGCCTTACTAGATTTCTATTT | 169 |
| | | ATTTGTAGCTCTCATTCATTGTTTT | 170 |
| | | CTTCTCTAGCCCAAACAGCGACATG | 171 |
| | | AGTCCCCTTCTTCAGAGTCAATAGA | 172 |
| | | AAGACCTGTTCACTAGCATTTTCAA | 173 |
| | | AAGGGGGTTCTAAAGCATTCAAGTG | 174 |
| | | AAATGACTTCTTAATTCCTGCCTTT | 175 |
| | | AATTCCTGCCTTTAGTGTCAACTTT | 176 |
| | | TACAGGTTTCAATTGTGGCATTAGG | 177 |
| | | GACTACATGAAATTGTGTGCCCCTA | 178 |
| | | AATCAGCTATAGCATCTTTCTAGAA | 179 |
| CLIC5 | 219866_at | GTTGATGCCAAAATACCCACGGGGT | 180 |
| | | TACCAGCCATGGGGTTTGCTTGCTT | 181 |
| | | CAGAGGTGATTACAGGCCTGGGTTT | 182 |
| | | GCCTGGGTTTGACTGTGCTTACCAA | 183 |
| | | TCTTTATGAGCCTCGATGTTCCCTG | 184 |
| | | AGGCCTTCTCTCATGATCTAAGTCT | 185 |
| | | AAGTCTTGGACTGGTGGCATCATGT | 186 |
| | | GGTGGCATCATGTAACTGCTAACCT | 187 |
| | | TCTGGAATGCAGGTCTGTCGGCTGG | 188 |
| | | TGCTCCTGCCTGATTCAACTGTAGC | 189 |
| | | GTCCATGAGACTTTCTGACTAGGAA | 190 |
| KLF4 | 221841_s_at | ATCCGACTTGAATATTCCTGGACTT | 191 |
| | | GCCAAGGGGGTGACTGGAAGTTGTG | 192 |
| | | GGAAGACCAGAATTCCCTTGAATTG | 193 |
| | | AAAGATCACCTTGTATTCTCTTTAC | 194 |
| | | GATGGTGCTTGGTGAGTCTTGGTTC | 195 |
| | | AAACTGCTGCATACTTTGACAAGGA | 196 |
| | | AATCTATATTTGTCTTCCGATCAAC | 197 |
| | | ATACCTGGTTTACTTCTTTAGCATT | 198 |
| | | CAGACAGTCTGTTATGCACTGTGGT | 199 |
| | | GGTTTATTCCCAAGTATGCCTTAAG | 200 |
| | | TTTTCTATATAGTTCCTTGCCTTAA | 201 |
| RTN4IP1 | 224509_s_at | GGAAGCTTGGTGCAGACGATGTAAT | 202 |
| | | GGCGGATCCACTGAAACATGGGCTC | 203 |
| | | ACATGGGCTCCAGATTTTCTCAAGA | 204 |
| | | GAAATGGTCAGGAGCCACCTATGTG | 205 |
| | | TATGTGACTTTGGTGACTCCTTTCC | 206 |
| | | TTCCTCCTGAACATGGACCGATTGG | 207 |
| | | GGCATGTTGCAGACAGGAGTCACTG | 208 |
| | | GAAAGGAGTCCATTATCGCTGGGCA | 209 |
| | | TATCGCTGGGCATTTTTCATGGCCA | 210 |
| | | GGCCAGTGGCCCATGTTTAGATGAC | 211 |
| | | GGAAAGATCCGGCCAGTTATTGAAC | 212 |
| H19 | 224997_x_at | CCTTCTGTCTCTTTGTTTCTGAGCT | 213 |
| | | CTTCTGTCTCTTTGTTTCTGAGCTT | 214 |
| | | TTCTGTCTCTTTGTTTCTGAGCTTT | 215 |
| | | TCTGTCTCTTTGTTTCTGAGCTTTC | 216 |
| | | CTGTCTCTTTGTTTCTGAGCTTTCC | 217 |
| | | TGTCTCTTTGTTTCTGAGCTTTCCT | 218 |
| | | TCTCTTTGTTTCTGAGCTTTCCTGT | 219 |
| | | GAAGCTCCGACCGACATCACGGAGC | 220 |
| | | AGCTCCGACCGACATCACGGAGCAG | 221 |
| | | CTCCGACCGACATCACGGAGCAGCC | 222 |
| | | TCACGGAGCAGCCTTCAAGCATTCC | 223 |
| PILRB | 225321_s_at | GGGATGTGTATTAGCCCCGGAGGAC | 224 |
| | | TAGCCCCGGAGGACGTGATGTGAGA | 225 |
| | | TGATGTGAGACCCGCTTGTGAGTCC | 226 |
| | | CACTCGTTCCCCATTGGCAAGATAC | 227 |
| | | TACATGGAGAGCACCCTGAGGACCT | 228 |
| | | GTCCCTGAATCACCGACTGGAGGAG | 229 |
| | | GAGTTACCTACAAGAGCCTTCATCC | 230 |
| | | CCAGGAGCATCCACACTGCAATGAT | 231 |
| | | AGGAATGAGGTCTGAACTCCACTGA | 232 |
| | | TGAACTCCACTGAATTAAACCACTG | 233 |
| | | GCAGTGCAAAGAGTTCCTTTATCCT | 234 |
| TET1 | 228906_at | CCACTCATCTACTCATTCTTCGAGT | 235 |
| | | GAGTCTACACTTATTGAATGCCTGC | 236 |
| | | GATCTCTCTCTCAATAGGTTTCTTA | 237 |
| | | TTGTGACGCTTGTTGCAGTTTACCA | 238 |
| | | AATGTTTCCATTCCGTTGTTGTAGT | 239 |
| | | TAAGCTGATTACCCCACTGTGGGAA | 240 |
| | | GGATTCCTACTTTGTTGGACTCTCT | 241 |
| | | TTGGACTCTCTTTCCTGATTTTAAC | 242 |
| | | TTTAACAATTTACCATCCCATTCTC | 243 |
| | | GTGATTGTATGCTGGCTACACTGCT | 244 |
| | | GCTACACTGCTTTTAGAATGCTCTT | 245 |
| ZSWIM7 | 229119_s_at | ATCTGTTATCGCTGAAGTTTCTCTT | 246 |
| | | CAGGCCTTGGACCTAGTTGATCGAC | 247 |
| | | TTGATCGACAGTCCATCACCTTAAT | 248 |
| | | CACCTTAATCTCATCACCCAGTGGA | 249 |
| | | GAAGGCGTGTTTACCAGGTCCTTGG | 250 |
| | | TTGGCTTCTTGTCATTACTGTTCAT | 251 |
| | | TACTGTTCATGTCCTGCATTTGCAT | 252 |
| | | GCATTTGCATTCTCAGTGCTACGGA | 253 |
| | | AAGCATCTCTTGGCAGTTTACCTGA | 254 |
| | | GAGAAGCCCTGTACAGTCTTGTCAA | 255 |
| | | AGCCAGTCTCTGAGACGCTTCGGTA | 256 |
| SMTNL2 | 229730_at | CCAGAGTTTTTTACTTCCTCACGCG | 257 |
| | | TCCTCACGCGATTGTAGGTTCCTCT | 258 |
| | | GAGACCGCTTAATCAGCAGCTTGAC | 259 |
| | | AACAGTTTAATCACTCCCAAGTCCT | 260 |
| | | CTGGGCAACAGATGACCTTCAAGTC | 261 |
| | | CCTCCGCTCTCCGGGGAGATGGGAA | 262 |
| | | GGGAGATGGGAAGGCTCTCCTCTCG | 263 |
| | | GAGGCCCCACAAGTGTTTGGCTAAG | 264 |
| | | TTGGCTAAGCACAGGCTCTCGGGAA | 265 |
| | | CAGGCTCTCGGGAATTTAACACTTT | 266 |
| | | GGGAAGGAATAGGCCCTTTGTGCTG | 267 |
| UNKL | 229908_s_at | CAAAGAATGGCTGGCAGCGCTGCCA | 268 |
| | | TCAGGGATGGCTCCTAGGTGGCTGA | 269 |
| | | CCTGTCGTCTGTAACTCTAGTGTTC | 270 |
| | | AACTCTAGTGTTCGACATTCGCCGT | 271 |
| | | GACATTCGCCGTGATACAGTGGTGT | 272 |
| | | TCCGCGTGGACGCCTCAAGTGGATT | 273 |
| | | CAAGTGGATTAATTTCTGGAAGCCT | 274 |
| | | TGGAAGCCTCAATCTGTATGTTTGA | 275 |
| | | AATCATTTACTTGTAGCGAACTGTT | 276 |
| | | TTTTTTACACTATAGCATTTATGCA | 277 |
| | | TGGTTTACAGAATTCATGGAGTTAT | 278 |
| SYPL2 | 230611_at | TATATTCACTCCTGCCAAGGACTCC | 279 |
| | | AGAGCAAGGAAGCCTCGTTCTCTTT | 280 |
| | | TTGATTTAGGCTACGGCCTCACTCT | 281 |
| | | ACTCTCTATGGCCACCCTAAGAGGA | 282 |
| | | TTCACCTCATTACCTCCAGAGGGCT | 283 |
| | | CTGGGCAGGGCCAAGTGCCTCATAG | 284 |
| | | GCCTCATAGGACTCATGTTCTCTCC | 285 |
| | | TGGGCAGGGTACTTGCCCTTTGTCC | 286 |
| | | CACCTAGGACCTTTCCTGGACATGA | 287 |
| | | GACATGAGTTTCCTTCACTATCATA | 288 |
| | | TCATAGTCATGAGCCTCCTACTTCT | 289 |
| BTNL9 | 230992_at | GGTCATCGAATCTGCATGCATCCCT | 290 |
| | | ATGCATCCCTCATACATCTGGAGAC | 291 |
| | | GAAGGTTCCAGAGTTACTGACTGAG | 292 |
| | | TGACTGAGATTTCTGAGCTTTTTTC | 293 |
| | | CTCCCAAACACATCGCTCCTTGGGG | 294 |
| | | ATCGCTCCTTGGGGTTACACTAGGT | 295 |
| | | ACTAGGTTTGTTTCCATCTGGCTTG | 296 |
| | | GGCTTGAGGCTATTTGCAGGCGAGA | 297 |
| | | GCAGGCGAGAGTGCAGAGTCTGTAA | 298 |
| | | CTGTAATGAACCTCCCAGATTCTCT | 299 |
| | | CAGATTCTCTGACGAAGGGGTCCCC | 300 |
| DIS3L | 235005_at | GTGGAAGAAGCTCAGCTTGCCCAAG | 301 |
| | | GAAGCTCAGCTTGCCCAAGAAGTCA | 302 |
| | | GGAATATCAAGAATATCGCCAAACA | 303 |
| | | GGGAAGGAGCCTATACACACTTCTA | 304 |
| | | GAGCCTATACACACTTCTAGAGGAG | 305 |
| | | GGAGATACGGGACCTAGCTCTCCTG | 306 |
| | | ATTTAATGTGTGTCACTCAGTGCTC | 307 |
| | | TGTCACTCAGTGCTCTAGTCGATCA | 308 |
| | | GTGCTCTAGTCGATCAGGACTGGGT | 309 |
| | | AGGACTGGGTAGCTATTTCGCATAT | 310 |
| | | GGGTAGCTATTTCGCATATATGTAA | 311 |
| FLJ43663/ | 238619_at | ACCAGCTACAGAGACGTTTCTTCCC | 312 |
| Pri-miR29 | | AAATCAAACTATCTTCTTCTCCTTA | 313 |
| | | TCTTCTCCTTAGCCGTTCAAATAGC | 314 |
| | | GAAATACACAGGCCTCTTTTCGTTT | 315 |
| | | GGCACATCATGCCTAGGTTGCTTTG | 316 |
| | | ATCACTTCCTCCTAAAGCAGTCTTA | 317 |
| | | GCATAGTCATAGTCTGTGATCTCAG | 318 |
| | | TGCTTCCTTCTAGAACATCTGAGTT | 319 |
| | | GACATCACTGGCCTTCAACAGGTGT | 320 |
| | | TGGATGGCCACAGATCATCCACCTG | 321 |
| | | ATCCACCTGCCAAACAGTTAACCCT | 322 |
| QRSL1 | 241933_at | CAGACACCACAACATCCTAGATGGA | 323 |
| | | CACACCTGGCCGAAATAATAATATT | 324 |
| | | ATTAAATCTCTTGTTCCTGTATCTC | 325 |
| | | GTTCCTGTATCTCTACATGAGCTGC | 326 |
| | | GTATCTCTACATGAGCTGCACTAAT | 327 |
| | | GAGCTGCACTAATAATTTGAATCTG | 328 |
| | | AAGTGAAACATTTACCGTTCTCATA | 329 |
| | | TACCGTTCTCATATACTGATACCCA | 330 |
| | | TACTGATACCCAACTACCATGAAAT | 331 |
| | | TTTTTACTCTTAATCTAGTAGGTCT | 332 |
| | | GTCACTGTCTGGGAATTTAAGTGGC | 333 |
| KCNQ5 | 244623_at | GAGTTTTTAAGTCCTGATCTGTTCT | 334 |
| | | GTCCTGATCTGTTCTAAGGTGCCTT | 335 |
| | | GTGATTCTGAAGTTCTTAATTTGCA | 336 |
| | | GGAAATCAGGCACAAATTGACCAAT | 337 |
| | | ATTGACCAATTCTCATGCCATTTGC | 338 |
| | | GGATGATGAAACCTGGCTAACTAAA | 339 |
| | | TATTAACTTGTCTCCCTAGAAGCTG | 340 |
| | | GAAGCTGAGATTTTTCGCCTTAAAT | 341 |
| | | TAAGTAAGCAGTTCTAAGTCATGTA | 342 |
| | | CAATGCAATTGTCTGTTTCCTGAAA | 343 |
| | | TTTGCTCTCTTTTACTGGGATTATT | 344 |
| ACTN4 | 244753_at | GACAGAGGGGAGCGGGGACAAGTTT | 345 |
| | | TTTTAAGTCTAAGCCTCCTGGGTGG | 346 |
| | | GTTTCAACATATGCTCCAGTCATGG | 347 |
| | | GCTCCAGTCATGGCAGACTTTGGCC | 348 |
| | | CAGCGCCCTTTTTCAGAGTGAACTG | 349 |
| | | TATCTGCCAGTGCTAGTTAGCAAAC | 350 |
| | | GCCCAAGGAATTTGAAACCGTTGAG | 351 |
| | | ACTTTCCGTTTTTGCTACACTGATT | 352 |
| | | GCTACACTGATTTATGTTGTGCTGG | 353 |
| | | TGTACAAGCCTTTGACCAGACCTTA | 354 |
| | | GTGACTTGCAAAAGCATTTTTACCT | 355 |

To validate this predictor set under diverse circumstances, it was tested in a blinded manner in an independent study. Affymetrix profiles were generated from pre-training muscle biopsy samples taken from Group 2 subjects (pre-intervention VO₂max=4.1±0.5 l/min), as described above. These young, physically active subjects underwent an intense interval-based aerobic training program. The sum of the expression of the 29 gene set (Σ29_{predict-RNA}; calculated as described above for Group 1) significantly correlated to the percent change in VO₂max in the blind validation group (FIG. 5; N=17, CC=0.51, p=0.02). A strong correlation was found between the molecular predictor of the first 29 gene set and the observed response to exercise as measured by change in VO₂max. In addition, three of the genes identified in Example 7 by quantitative trait locus (“QTL”) genotyping and candidate gene studies in Group 3 subjects (SVIL, NRP2 and MIPEP) to have a significant association with exercise were also used in the validation RNA data set (Group 2, FIG. 6). Addition of the expression levels of two of these validated genes, SVIL and NRP2, was found to improve the performance of the Gene Predictor Score (CC=0.64, p=0.009), while addition of MIPEP did not alter this improved performance.

Thus using the second independent study group, the predictor gene set was demonstrated to apply to human subjects with a wide range in aerobic fitness capacities and confirmed the validity of the gene selection process.

To use this Gene Predictor Score to predict the response of an individual, using the pain-free fine-needle method [26], a micro-muscle sample can be obtained (1-2 mg). Then, RNA will be isolated from the subject, and analyzed using a microarray for the expression of the 29 predictor gene set. The expression signal obtained from each predictor gene will be summed to produce an overall score. This score will then be related to the known relationship with aerobic fitness adaptation, and the subject will be classified into 4 broad categories.

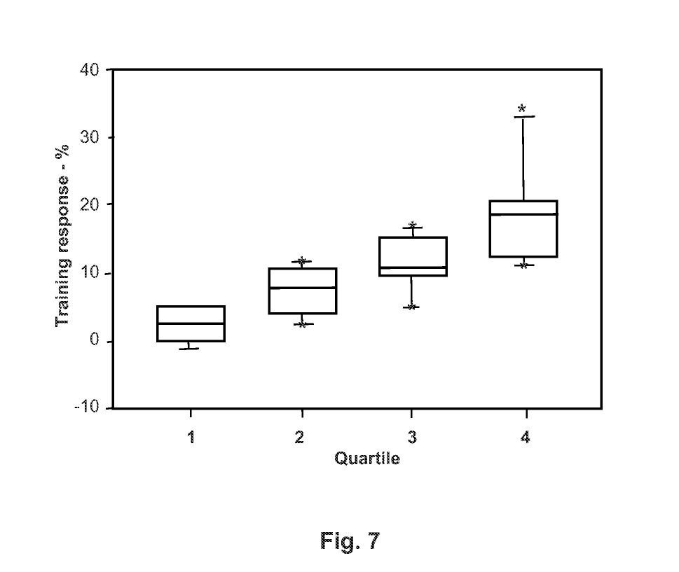

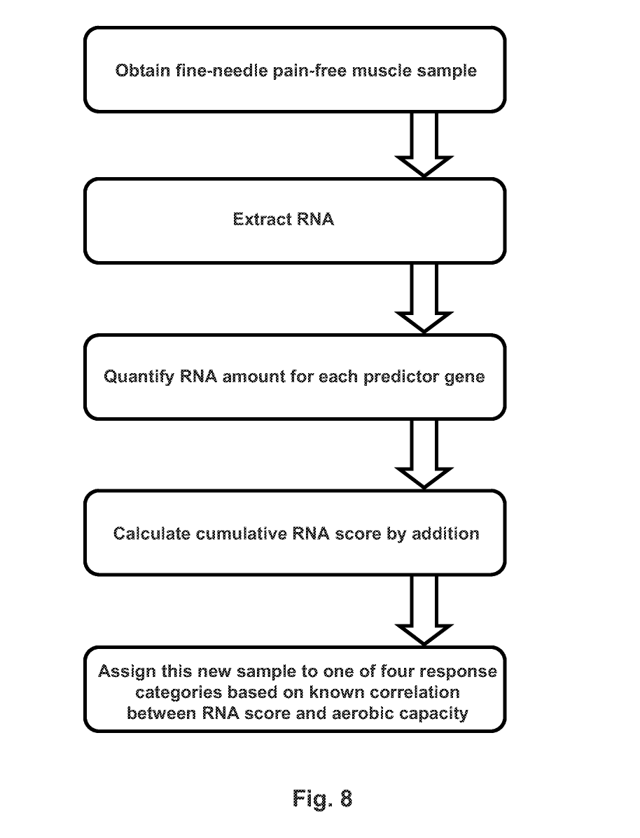

FIG. 7 is a summary of the performance of the predictor gene set across the entire RNA cohort of both Groups 1 and 2. The range of RNA based gene predictor scores has been split into quartiles. The 1st quartile represents the lowest sum of the 29 RNA gene expression values. Using this gene expression score, a subject can be classified as belonging to one of four categories, 1) non-responder; 2) poor responder; 3) good responder; and 4) high responder. FIG. 8 is a flow chart of one way a subject could be classified into one of the four groups in FIG. 7. This method is a simple way to classify a subject who is a non-responder or a high responder. The relative position of the score on this scale, based on reading from a regression line through the data, will predict general aerobic fitness potential.

### Example 7

### DNA SNP Based Biomarkers for Response to Exercise

A new analysis of the HERITAGE Family Study (n=473) was carried out using ˜300 tag SNPs for the 29 predictor gene probe-sets. A customized array for identified SNPs was typically made by Illumina by using sequences 60 base pairs (bp) on each side of a SNP. Sedentary subjects from 99 nuclear families were trained for 20 weeks with a fully standardized and monitored exercise program. The mean gain in maximal VO₂ was similar to that seen in the studies above (˜400 ml O₂), with a standard deviation of ˜200 ml O₂. Using a model fitting procedure, the heritability of the change in VO₂max was calculated to be about 47% [6], and thus genetic variants could, at most, expect to capture ˜50% of the total variance in the gain in maximal aerobic capacity. Six genes were identified from the predictor gene set that harboured genetic variants associated with gains in aerobic capacity (p<0.01 for each). When comparing the upper versus the lower quartile of the VO₂max response distribution, SNPs in SMTNL2, DEPDC6, SLC22A3, METTL3 and BTNL9 were found to differ the most in genotype or allele frequencies. In addition, in the comparison of the VO₂max response by genotype for the entire HERITAGE population, a variant in ID3 was also seen (rs11574; p=0.0058). ID3 is a TGFβ1 and superoxide-regulated gene, which interacts [27] with another member of the baseline predictor, KLF4, and appears essential for angiogenesis [28]. The imprinted transcript, SLC22A3 (OCT3), which harboured genetic variation associated with training response (p=0.0047), is part of the Air non-coding RNA imprinted locus mechanism, which interacts [29] with another of the predictor genes, H19. This suggests the predictor genes may participate in the regulation of imprinting, and that the mechanisms which link aerobic capacity and cardiovascular-metabolic disease may share common features with developmental processes [30, 31].

The SNPs that showed the strongest association with residual VO₂max are listed in Table 5. Table 5 also lists the two alleles at each SNP, and the base pair location of the SNP in the sequences used for the array. The actual sequences are found in the attached Sequence Listing. One gene, ACE, is not a SNP, but is an insertion/deletion of 289 bp. The ACE genotype was not found to be one of the final predictor 11 SNPs.

TABLE 5
SNPs set used in stepwise regression models described above. SNPs
(n = 35) showing strongest association with the changes in VO2mx
from ALL genes were selected.

| | | | | |
|---|---|---|---|---|
| | | | | SEQ ID NO: |
| | | | | (allele; bp |
| GENE | CHR | MAP | ALLELES | of SNP) |

A. HERITAGE genes and SNPs chosen for regression models (n = 10).

| | | | | | |
|---|---|---|---|---|---|
| | SNP* | | | | |
| SLC4A5 | rs828902 | 2 | 74,323,642 | C/T | 1 (C; 201) |
| TTN | rs10497520 | 2 | 179,353,100 | A/G | 2 (A; 61) |
| NRP2 | rs3770991 | 2 | 206,363,984 | A/G | 3 (A; 61) |
| CREB1 | rs2709356 | 2 | 208,120,337 | A/G | 4 (A; 61) |
| PPARD | rs2076167 | 6 | 35,499,765 | A/G | 5 (A; 256) |
| SVIL | rs6481619 | 10 | 30,022,960 | A/C | 6 (A; 61) |
| KIF5B | rs806819 | 10 | 32,403,990 | A/C | 7 (A; 61) |
| ACTN3 | rs1815739 | 11 | 66,084,671 | C/T | 8 (C; 293) |
| MIPEP | rs7324557 | 13 | 23,194,862 | A/G | 9 (A; 61) |
| ACE | Insertion | 17 | 58,919,622 | | 10 |
| | Deletion | 17 | | | 11 |

B. Molecular predictor genes and SNPs chosen for regression models
(n = 25).

| | | | | | |
|---|---|---|---|---|---|
| | SNP | | | | |
| ID3 | rs11574 | 1 | 23,758,085 | A/G | 12 (A; 61) |
| MAST2 | rs2236560 | 1 | 46,268,021 | A/G | 13 (A; 61) |
| SYPL2 | rs12049330 | 1 | 109,832,711 | A/C | 14 (A; 61) |
| SCN3A | rs7574918 | 2 | 165,647,425 | A/C | 15 (A; 61) |
| AMOTL2 | rs13322269 | 3 | 135,569,834 | A/G | 16 (A; 61) |
| BTNL9 | rs888949 | 5 | 180,425,011 | A/G | 17 (A; 61) |
| KCNQ5 | rs10943075 | 6 | 73,776,703 | A/G | 18 (A; 61) |
| RTN4IP1/ | rs898896 | 6 | 107,169,855 | A/G | 19 (A; 61) |
| QRSL1 | | | | | |
| SLC22A3 | rs2457571 | 6 | 160,754,818 | A/G | 20 (A; 61) |
| CPVL | rs4257918 | 7 | 29,020,374 | A/G | 21 (A; 61) |
| PILRB | rs13228694 | 7 | 99,778,243 | A/G | 22 (A; 61) |
| DEPDC6 | rs7386139 | 8 | 121,096,600 | A/G | 23 (A; 61) |
| KLF4 | rs4631527 | 9 | 109,309,857 | A/G | 24 (A; 61) |
| TET1 | rs12413410 | 10 | 70,055,236 | A/G | 25 (A; 61) |
| BTAF1 | rs2792022 | 10 | 93,730,409 | A/G | 26 (A; 61) |
| H19 | rs2251375 | 11 | 1,976,072 | A/C | 27 (A; 61) |
| METTL3 | rs1263809 | 14 | 21,058,740 | A/C | 28 (A; 61) |
| DIS3L | rs1546570 | 15 | 64,382,829 | A/C | 29 (A; 61) |
| UNKL | rs3751894 | 16 | 1,426,876 | A/G | 30 (A; 61) |
| IL32 | rs13335800 | 16 | 3,052,198 | A/T | 31 (A; 61) |
| SMTNL2 | rs7217556 | 17 | 4,425,585 | A/G | 32 (A; 61) |
| ZSWIM7 | rs10491104 | 17 | 15,825,286 | A/G | 33 (A; 61) |
| ENOSF1 | rs3786355 | 18 | 671,962 | A/G | 34 (A; 61) |
| IER2 | rs892020 | 19 | 13,128,185 | A/C | 35 (A; 61) |
| DNAJB1 | rs4926222 | 19 | 14,488,050 | A/G | 36 (A; 61) |

*ACE is not a SNP, but an insertion/deletion of 289 bp.

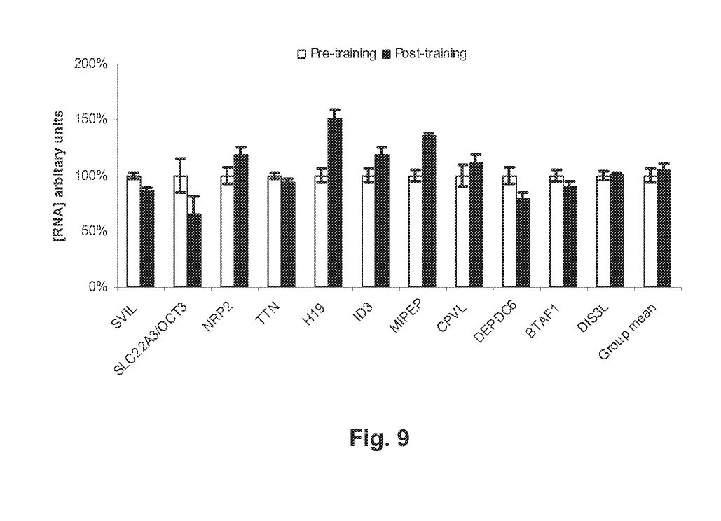

Utilizing 25 relevant genetic variants identified from the molecular predictor (n=25; Table 5B) and 10 from ongoing QTL and candidate gene studies within the HERITAGE project (n=10; Table 5A), a stepwise regression model was applied using the residual VO₂max responses, adjusted for major confounding variables, e.g., age, sex, baseline body weight, and baseline VO₂max. The results were striking: 11 SNPs captured 23% of the total variance in aerobic capacity responses (Table 6). Reciprocal analysis—genotype analysis back to expression variation—of the HERITAGE derived gene and SNPs, independently validated three genes. Thus addition of SVIL and NRP2 yielded an improved correlation coefficient (CC=0.60) and stronger p-value (p=0.009) for the validation data set (Group 2, FIG. 6) while MIPEP expression was negatively correlated (CC=−0.64, p=0.0051) and did not worsen or improve the performance of tissue based classifier. Finally, in support of the idea that the genotype-transcript associations are driven by genetic variation largely independent of environmental variables, expression of the genes that captured almost 50% of the total heritable variance was remarkably independent of exercise level, and the genes did not belong to the initial TRT (genes in FIGS. 3a and 3b, compared to those in FIG. 9).

TABLE 6
Stepwise Regression model for standardized residuals* of VO₂ₘₐₓ
training response in the HERITAGE Family Study.

| | | | RNA | | | | |
|---|---|---|---|---|---|---|---|
| Gene | | | level | | | | |
| (SNP; | | | stable | | | | |
| SEQ ID | Identification | RNA level | to | Genomic | partial | model | |
| NO;) | method | correlation | exercise | Location | r² | r² | p value |
| SVIL | QTL | YES (+) | YES | 10p11.2 | 0.0411 | 0.0411 | <.0001 |
| (rs6481619; | | | | | | | |
| 6) | | | | | | | |
| SLC22A3 | RNA | YES (+) | YES | 6q26-q27 | 0.0307 | 0.0718 | 0.0003 |
| (rs2457571; | predictor | | | | | | |
| 20) | | | | | | | |
| NRP2 | QTL | YES (+) | YES | 2q33.3 | 0.0224 | 0.0942 | 0.0017 |
| (rs3770991; | | | | | | | |
| 3) | | | | | | | |
| TTN | QTL | NO | YES | 2q31 | 0.0204 | 0.1146 | 0.0025 |
| (rs10497520; | | | | | | | |
| 2) | | | | | | | |
| H19 | RNA | YES (+) | NO | 11p15.5 | 0.0268 | 0.1414 | 0.0004 |
| (rs2251375; | predictor | | | | | | |
| 27) | | | | | | | |
| ID3 | RNA | YES (+) | YES | 1p36.13-p36.12 | 0.02 | 0.1615 | 0.0021 |
| (rs11574; | predictor | | | | | | |
| 12) | | | | | | | |
| MIPEP | QTL | YES (−) | YES | 13q12 | 0.0163 | 0.1778 | 0.0051 |
| (rs7324557; | | | | | | | |
| 9) | | | | | | | |
| CPVL | RNA | YES (+) | YES | 7p15-p14 | 0.0179 | 0.1957 | 0.0031 |
| (rs4257918; | predictor | | | | | | |
| 21) | | | | | | | |
| DEPDC6 | RNA | YES (+) | YES | 8q24.12 | 0.0112 | 0.2069 | 0.0185 |
| (rs7386139; | predictor | | | | | | |
| 23) | | | | | | | |
| BTAF1 | RNA | YES (+) | YES | 10q22-q23 | 0.0125 | 0.2194 | 0.0122 |
| (rs2792022; | predictor | | | | | | |
| 26) | | | | | | | |
| DIS3L | RNA | YES (+) | YES | 15q22.31 | 0.0095 | 0.2289 | 0.0279 |
| (rs1546570; | predictor | | | | | | |
| 29) | | | | | | | |

The SNPs and genes in Table 6 are given in the standard nomenclature adopted by the National Center of Biotechnology Information (NCBI). The sequence data for both the SNPs and genes listed are known and readily available from published databases, e.g., the NCBI dbSNP and OMIM databases. The sequence used in the genotyping array for each SNP listed in Table 5 is given in the attached Sequence Listing. Using the SNPs in Table 6 a scoring system was established for each allele based on gains in VO2max across the genotypes of predictor SNPs. The allele associated with the lowest gain was coded as 0 in the homozygotes while the heterozygotes were scored as one, and the homozygotes for the allele associated with the highest gain were scored as two. Table 7 sets out the scoring for the 11 SNPs.

TABLE 7
Scoring Scheme for the 11 SNPs

| | | Number of | Mean gain | |
|---|---|---|---|---|
| Gene | SNP | subjects | in VO2max | Score |
| SVIL | rs6481619 | | | |
| | A/A | 225 | 370 | 0 |
| | A/C | 193 | 413 | 1 |
| | C/C | 24 | 536 | 2 |
| SLC22A3 | rs2457571 | | | |
| | A/A | 109 | 365 | 0 |
| | A/G | 246 | 384 | 1 |
| | G/G | 117 | 451 | 2 |
| NRP2 | rs3770991 | | | |
| | A/A | 4 | 440 | 2 |
| | A/G | 97 | 461 | 1 |
| | G/G | 402 | 380 | 0 |
| TTN | rs10497520 | | | |
| | A/A | 8 | 339 | 0 |
| | A/G | 89 | 334 | 1 |
| | G/G | 375 | 412 | 2 |
| H19 | rs2251375 | | | |
| | A/A | 47 | 353 | 0 |
| | A/C | 173 | 376 | 1 |
| | C/C | 252 | 418 | 2 |
| ID3 | rs11574 | | | |
| | A/A | 23 | 367 | 0 |
| | A/G | 178 | 372 | 1 |
| | G/G | 271 | 414 | 2 |
| MIPEP | rs7324557 | | | |
| | A/A | 54 | 430 | 2 |
| | A/G | 191 | 410 | 1 |
| | G/G | 226 | 377 | 0 |
| CPVL | rs4257918 | | | |
| | A/A | 11 | 291 | 0 |
| | A/G | 120 | 369 | 1 |
| | G/G | 341 | 409 | 2 |
| DEPDC6 | rs7386139 | | | |
| | A/A | 328 | 416 | 2 |
| | A/G | 129 | 349 | 1 |
| | G/G | 15 | 372 | 0 |
| BTAF1 | rs2792022 | | | |
| | A/A | 247 | 382 | 0 |
| | A/G | 185 | 414 | 1 |
| | G/G | 39 | 406 | 2 |
| DIS3L | rs1546570 | | | |
| | A/A | 31 | 416 | 2 |
| | A/C | 174 | 418 | 1 |
| | C/C | 267 | 379 | 0 |

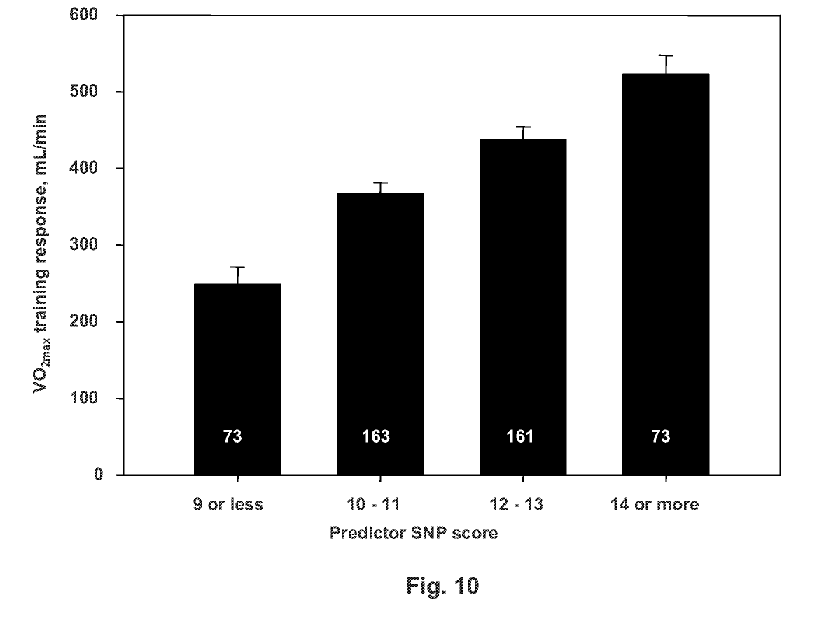

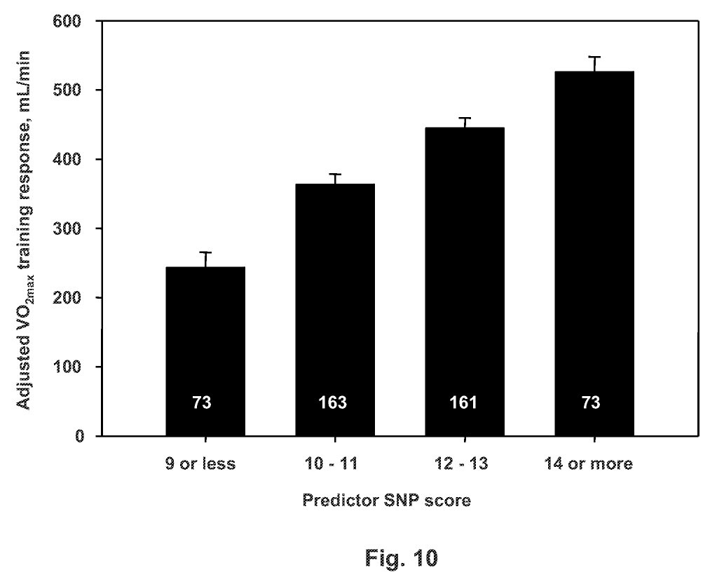

Using the above scoring method, each subject in Group 3 was given a score for each SNP, and then the scores were added for a total Predictor SNP score. The Predictor SNP scores were assigned to one of four catEgories of response to exercise based on the mean VO₂max for the subjects in the group: ≦9, low responders; 10-11, less than average responder; 12-13, greater than average responder; and ≧14, high responder. FIG. 10 shows the results of applying the Predictor SNP scores to the HERITAGE Study group, and shows the mean VO2max training response for the individuals assigned to each category by the Predictor SNP score. FIG. 11 shows similar results, but uses an adjusted mean VO2max training response (adjusted for age, sex, baseline body weight and baseline VO2max).

As shown above, the above 11 SNPs can be used to predict the response to exercise in a human subject. A DNA sample can easily be obtained from saliva, cheek cells, or other body fluid or cells. This sample can be assayed using techniques commonly used in the field for the allele present at each locus of each SNP. This allele distribution in the subject can then be scored using the system described above to determine the predicted ability to respond to exercise. With all 11 SNPs, the scoring can occur as shown above with the reference categories defined above.

The predictive gene sets and SNP markers used in the prototype experiments described above were based on three groups that were all ethnically Caucasian. While we have no reason to expect substantially different results in individuals of other ethnicities, neither do we yet have corresponding data. If such differences should exist, then a person of ordinary skill in the art may readily, following the teachings of this description, identify those differences and make any appropriate modifications to the sequences and markers used in the techniques described.

### REFERENCES

1. Blair S N, Kampert J B, Kohl H W, 3rd, Barlow C E, Macera C A, Paffenbarger R S, Jr., Gibbons L W: Influences of cardiorespiratory fitness and other precursors on cardiovascular disease and all-cause mortality in men and women. JAMA 1996, 276(3):205-210.
2. Blair S N, Kohl H W, 3rd, Paffenbarger R S, Jr., Clark D G, Cooper K H, Gibbons L W: Physical fitness and all-cause mortality. A prospective study of healthy men and women. Jama 1989, 262(17):2395-2401.
3. Gulati M, Pandey D K, Arnsdorf M F, Lauderdale D S, Thisted R A, Wicklund R H, Al-Hani A J, Black H R: Exercise capacity and the risk of death in women: the St James Women Take Heart Project. Circulation 2003, 108(13):1554-1559.
4. Kokkinos P, Myers J, Kokkinos J P, Pittaras A, Narayan P, Manolis A, Karasik P, Greenberg M, Papademetriou V, Singh S: Exercise capacity and mortality in black and white men. Circulation 2008, 117(5):614-622.
5. Myers J, Prakash M, Froelicher V, Do D, Partington S, Atwood J E: Exercise capacity and mortality among men referred for exercise testing. N Engl J Med 2002, 346(11):793-801.
6. Bouchard C, An P, Rice T, Skinner J S, Wilmore J H, Gagnon J, Perusse L, Leon A S, Rao D C: Familial aggregation of VO(2max) response to exercise training: results from the HERITAGE Family Study. J Appl Physiol 1999, 87(3):1003-1008.
7. Vollaard N B, Constantin-Teodosiu D, Fredriksson K, Rooyackers O E, Jansson E, Greenhaff P L, Timmons J A, Sundberg C J: Systematic analysis of adaptations in aerobic capacity and submaximal energy metabolism provides a unique insight into determinants of human aerobic performance. J Appl Physiol 2009.
8. Saltin B, Calbet J A: Point: in health and in a normoxic environment, VO2 max is limited primarily by cardiac output and locomotor muscle blood flow. J Appl Physiol 2006, 100(2):744-745.
9. Hamilton M T, Booth F W: Skeletal muscle adaptation to exercise: a century of progress. J Appl Physiol 2000, 88(1):327-331.
10. Timmons J A, Larsson O, Jansson E, Fischer H, Gustafsson T, Greenhaff P L, Ridden J, Rachman J, Peyrard-Janvid M, Wahlestedt C et al: Human muscle gene expression responses to endurance training provide a novel perspective on Duchenne muscular dystrophy. Faseb J 2005, 19(7):750-760.
11. Frazer K A, Murray S S, Schork N J, Topol E J: Human genetic variation and its contribution to complex traits. Nat Rev Genet 2009, 10(4):241-251.
12. Snyder M, Weissman S, Gerstein M: Personal phenotypes to go with personal genomes. Mol Syst Biol 2009, 5:273.
13. Chen W W, Li L, Yang G Y, Li K, Qi X Y, Zhu W, Tang Y, Liu H, Boden G: Circulating FGF-21 levels in normal subjects and in newly diagnose patients with Type 2 diabetes mellitus. Exp Clin Endocrinol Diabetes 2008, 116(1):65-68.
14. Knudsen S, Knudsen S: Guide to analysis of DNA microarray data, 2nd edn. Hoboken, N.J.: Wiley-Liss; 2004.
15. Timmons J A, Gustafsson T, Sundberg C J, Jansson E, Greenhaff P L: Muscle acetyl group availability is a major determinant of oxygen deficit in humans during submaximal exercise. Am J Physiol 1998, 274(2 Pt 1):E377-380.
16. Bouchard C, Leon A S, Rao D C, Skinner J S, Wilmore J H, Gagnon J: The HERITAGE family study. Aims, design, and measurement protocol. Med Sci Sports Exerc 1995, 27(5):721-729.
17. Bouchard C, Rankinen T, Chagnon Y C, Rice T, Perusse L, Gagnon J, Borecki I, An P, Leon A S, Skinner J S et al: Genomic scan for maximal oxygen uptake and its response to training in the HERITAGE Family Study. J Appl Physiol 2000, 88(2):551-559.
18. Choe S E, Boutros M, Michelson A M, Church G M, Halfon M S: Preferred analysis methods for Affymetrix GeneChips revealed by a wholly defined control dataset. Genome Biol 2005, 6(2):R16.
19. Larsson O, Wahlestedt C, Timmons J A: Considerations when using the significance analysis of microarrays (SAM) algorithm. BMC Bioinformatics 2005, 6(1):129.
20. Li C, Hung Wong W: Model-based analysis of oligonucleotide arrays: model validation, design issues and standard error application. Genome Biol 2001, 2(8):RESEARCH0032.
21. Li C, Wong W H: Model-based analysis of oligonucleotide arrays: expression index computation and outlier detection. Proc Natl Acad Sci USA 2001, 98(1):31-36.
22. Saxena R, de Bakker P I, Singer K, Mootha V, Burtt N, Hirschhorn J N, Gaudet D, Isomaa B, Daly M J, Groop L et al: Comprehensive association testing of common mitochondrial DNA variation in metabolic disease. Am J Hum Genet 2006, 79(1):54-61.
23. Abecasis G R, Cardon L R, Cookson W O: A general test of association for quantitative traits in nuclear families. Am J Hum Genet 2000, 66(1):279-292.
24. Tusher V G, Tibshirani R, Chu G: Significance analysis of microarrays applied to the ionizing radiation response. Proc Natl Acad Sci USA 2001, 98(9):5116-5121.
25. Keller P, Vollaard N J B, Babraj J, Ball D, Sewell D A, Timmons J A: Using systems biology to define the essential biological networks responsible for adaptation to endurance exercise training. Biochem Soc Trans 2007.
26. Nickenig G, Baudler S, Muller C, Werner C, Werner N, Welzel H, Strehlow K, Bohm M: Redox-sensitive vascular smooth muscle cell proliferation is mediated by GKLF and Id3 in vitro and in vivo. Faseb J 2002, 16(9):1077-1086.
27. Lyden D, Young A Z, Zagzag D, Yan W, Gerald W, O'Reilly R, Bader B L, Hynes R O, Zhuang Y, Manova K et al: Id1 and Id3 are required for neurogenesis, angiogenesis and vascularization of tumour xenografts. Nature 1999, 401(6754):670-677.
28. Nagano T, Mitchell J A, Sanz L A, Pauler F M, Ferguson-Smith A C, Feil R, Fraser P: The Air noncoding RNA epigenetically silences transcription by targeting G9a to chromatin. Science 2008, 322(5908):1717-1720.
29. Gluckman P D, Hanson M A: Developmental plasticity and human disease: research directions. J Intern Med 2007, 261(5):461-471.
30. van Hoek M, Langendonk J G, de Rooij S R, Sijbrands E J, Roseboom T J: A Genetic Variant in the IGF2BP2 Gene may Interact with Fetal Malnutrition on Glucose Metabolism. Diabetes 2009.

The complete disclosures of all references cited in this specification are hereby incorporated by reference. In the event of an otherwise irreconcilable conflict, however, the present specification shall control.

What is claimed:

1. A method for predicting a characteristic of a human subject; said method comprising assaying a DNA or RNA sample from the subject for the presence or absence of one or more single nucleotide polymorphisms selected from the group consisting of the SNPs located at the locus represented by position 61 of each of the sequences of SEQ ID NO: 6 (SVIL), SEQ ID NO: 20 (SLC22A3), SEQ ID NO: 3 (NRP2), SEQ ID NO: 2 (TTN), SEQ ID NO: 27 (H19), SEQ ID NO: 12 (ID3), SEQ ID NO: 9 (MIPEP), SEQ ID NO: 21 (CPVL), SEQ ID NO: 23 (DEPDC6), SEQ ID NO: 26 (BTAF1), and SEQ ID NO: 29 (DIS3L); and correlating any such single nucleotide polymorphisms thus identified in the subject to the characteristic; wherein the characteristic is selected from the group consisting of: (a) the expected response of the subject's maximal oxygen uptake to an aerobic exercise program, (b) the expected response of the subject's aerobic capacity to an aerobic exercise program, and (c) the subject's risk of cardiovascular disease.

2. The method of claim 1, wherein the characteristic is the expected response of the subject's maximal oxygen uptake to an aerobic exercise program.

3. The method of claim 1, wherein the characteristic is the expected response of the subject's aerobic capacity to an aerobic exercise program.

4. The method of claim 1, wherein the characteristic is the subject's risk of cardiovascular disease.

5. The method of claim 1, wherein the method comprises assaying the DNA or RNA sample for the presence or absence of five or more single nucleotide polymorphisms as recited.

6. The method of claim 1, wherein the method comprises assaying the DNA or RNA sample for the presence or absence of eleven or more single nucleotide polymorphisms as recited.

7. The method of claim 1, wherein the method comprises assaying the DNA or RNA sample for the presence or absence of all of the single nucleotide polymorphisms as recited.

8. A method for predicting a characteristic of a human subject; said method comprising assaying an RNA sample from the subject for the level of transcription of one or more RNAs selected from the group consisting of SEQ ID NOs: 37-355; and correlating the level of transcription of the one or more RNAs to the characteristic; wherein the characteristic is selected from the group consisting of: (a) the expected response of the subject's maximal oxygen uptake to an aerobic exercise program, (b) the expected response of the subject's aerobic capacity to an aerobic exercise program, and (c) the subject's risk of cardiovascular disease.

9. The method of claim 8, wherein the characteristic is the expected response of the subject's maximal oxygen uptake to an aerobic exercise program.

10. The method of claim 8, wherein the characteristic is the expected response of the subject's aerobic capacity to an aerobic exercise program.

11. The method of claim 8, wherein the characteristic is the subject's risk of cardiovascular disease.

12. The method of claim 8, wherein the method comprises assaying the RNA sample for the presence or absence of fifty or more RNAs as recited.

13. The method of claim 8, wherein the method comprises assaying the RNA sample for the presence or absence of 100 or more RNAs as recited.

14. The method of claim 8, wherein the method comprises assaying the RNA sample for the presence or absence of all of the RNAs as recited.

15. A method for identifying one or more single nucleotide polymorphisms in a human subject as markers that are correlated to the expected level of response by the subject to an intervention; said method comprising the steps of: (a) selecting a group of human subjects; (b) collecting an RNA sample from each of the subjects; (c) assaying each of the RNA samples for the level of transcription of RNA for 10 or more different genes; (d) exposing each of the subjects to the intervention after collecting the RNA samples; (e) determining the response level of each subject to the intervention; (f) identifying individuals within the group having a high response to the intervention and individuals within the group having a low response to the intervention; (g) correlating the assayed RNA levels to high response, or to low response; (h) validating the hypothesized correlations by repeating steps (a) through (g) in at least one additional group of human subjects; and correlating genomic single nucleotide polymorphisms in the DNA coding sequences to the corresponding RNAs correlated to high response, or to low response, or both.

16. A kit comprising single-stranded nucleic acids that are complementary to or identical to at least 5 consecutive nucleotides around the single nucleotide polymorphisms of five or more sequences selected from the group consisting of the single nucleotide polymorphisms listed in Table 6 of the specification.

17. A kit comprising 100 or more of single-stranded nucleic acids that are complementary to or identical to at least 5 consecutive nucleotides around the sequences selected from the group consisting of the sequences listed in Table 4 of the specification.
